(19) United States
(12) Patent Application Publication
(10) Pub. No.: US 2018/0299445 A1
(43) Pub. Date: Oct. 18, 2018

(54) BIOMARKERS AND METHODS OF USING SAME

(71) Applicant: BioDetego LLC, Philadelphia, PA (US)

(72) Inventors: Anthony P. Shuber, Northbridge, MA (US); David S. Zuzga, Collingswood, NJ (US)

(21) Appl. No.: 15/944,079

(22) Filed: Apr. 3, 2018

Related U.S. Application Data
(60) Provisional application No. 62/540,862, filed on Aug. 3, 2017.
(60) Provisional application No. 62/480,871, filed on Apr. 3, 2017.

(51) Int. Cl. G01N 33/574

(52) U.S. Cl. CPC G01N 33/574

(57) ABSTRACT

The present disclosure provides biomarkers and methods for providing a clinical assessment of a subject. In particular, the present disclosure provides methods for measuring at least one biomarker to classifying a subject as being test negative, with a high negative predictive value, and recommending that test negative subject be excluded from treatment. The present disclose also provides methods for measuring at least one biomarker in a subject not excluded from treatment and classifying a subject as being test positive, with a high positive predictive value, and recommending that test positive subject receive treatment.

### CROSS-REFERENCE TO RELATED APPLICATIONS

This application claims priority to, and the benefit of, U.S. Ser. No. 62/480,871, filed Apr. 3, 2017 and U.S. Ser. No. 62/540,862, filed Aug. 3, 2017. The contents of each application is incorporated by reference in its entirety.

### INCORPORATION-BY-REFERENCE OF SEQUENCE LISTING

The contents of the file named “BIDE-001_001US_SequenceListing_ST25”, which was created on Jun. 8, 2018, and is 6.64 KB in size are hereby incorporated by reference in their entirety.

### BACKGROUND OF THE INVENTION

Standard screening assays are used by clinicians to assess the current health status of patients and to provide insight into the patient's risk of having a particular disease or condition. Screening assays generally employ a threshold above which a patient is screened as “positive” for the indicated disease and below which the patient is screened as “negative” for the indicated disease. Thresholds in screening assays generally are chosen in order to maximize the number of patients who will receive further intervention in the form of diagnostic monitoring or therapy. However, all screening assays result in false positive and false negative determinations. This means that there is a portion of the screened patient population who are screened positive and prescribed further intervention who, in fact, are negative and do not need further intervention. There are also patients who are screened negative but who are actually positive and require accelerated or more significant (i.e. relative to standard of care) intervention. In standard screening assays, patients cannot be unambiguously placed into any clinical category. Thus, there is always a population of patients (false negatives and false positives) who are referred from improper follow-up due to the ambiguity inherent in screening.

An area in which this ambiguity has particular significance for patients is that of recurrence monitoring in cancer. Cancer patients who have been successfully treated must be concerned that either a primary tumor will recur or a secondary tumor will develop as a result of chemotherapy and radiation used to eradicate the original cancer. Screening those patients for recurrence is important, as many recurrent cancers can be treated with minimal intervention if caught early enough. Thus, most cancer survivors are monitored on intervals that depend primarily upon the type of cancer for which they were originally treated and the type of original treatment they received. Screening assays for recurrent cancer apply the same statistical criteria as do cholesterol screening and other common assays. Thus, there are a significant number of patients whose assay scores are ambiguous because they are at or near the limit of a range for “normal” samples. Those patients typically are required to continue screening and perhaps are even subjected to prophylaxis that may be unnecessary but is prescribed by the prevailing standard of care. Moreover, patients who are at increased risk of recurrence often are not identified as such and therefore are not provided with increased surveillance that may be necessary to effect early detection of recurrence.

There is, therefore, a need in the art to provide screening assays that eliminate as many ambiguous results as possible, thereby limiting the number of patients who must endure unnecessary procedures and optimizing identification of patients who would certainly benefit from continual monitoring and/or intervention. The present disclosure addresses these needs in the art.

### SUMMARY OF THE INVENTION

The present disclosure provides a method for providing a clinical assessment of a subject in need thereof including a) measuring the amount of at least one first biomarker in at least one first sample from the subject to generate a first score; b) comparing the first score to a first predetermined threshold to determine if the subject is test positive or test negative, wherein the predetermined threshold has a negative predictive value of at least 80%; c) providing a clinical assessment, wherein if the subject is determined to be test negative, the clinical assessment comprises recommending the subject is excluded from treatment; d) measuring the amount of at least one second biomarker in at least one second sample from a subject determined to be test positive in step (b) to generate a second score; e) comparing the second score to a second predetermined threshold to determine if the subject is test positive for the second predetermined threshold, wherein the predetermined threshold has a positive predictive value of at least 40%; and f) providing a clinical assessment, wherein if the subject is determined to be test positive in step (e), the clinical assessment comprises recommending the subject receive treatment.

The methods of the present disclosure can also include administering a treatment including chemotherapy, immunotherapy, radiotherapy, or a combination thereof, to a subject recommended to receive treatment.

The at least one first biomarker and the at least one second biomarker can be the same or can be different. The at least one first biomarker or the at least one second biomarker can be an amino acid molecule, a protein, a polypeptide, a nucleic acid molecule, DNA, RNA, a lipid, a carbohydrate, or a combination thereof.

The at least one first sample and the at least one second sample can be the same or can be different. The at least first sample or the at least second sample can be any cell, tissue or bodily fluid. For example the at least first sample or the at least second sample can be a tumor tissue, normal tissue, normal tissue adjacent to a tumor, saliva, plasma, blood, serum, spinal fluid, lymphatic fluid, urine, or a combination thereof. In some aspects, the at least first sample or the at least second sample is tumor tissue. In particular, an epithelial tumor tissue. In some aspects, the at least first sample or the at least second sample is a tumor tissue and normal tissue adjacent to that tumor tissue.

The first score and the second score can be the same or can be different. The first score, the second score, or both the first score and the second score, can be calculated using an algorithm. The first predetermined threshold and the second predetermined threshold can be the same or can be different. The first predetermined threshold, the second predetermined threshold, or both the first predetermined threshold and the second predetermined threshold, can be calculated using an algorithm.

In one aspect, the algorithm that is used to calculate the score is selected from (CA157/CAvasp)/(NAT157/NATvasp); (CA157/CAvasp); SQRT(CA239/NAT239×CA157/NAT157)/(CAvasp/NATvasp)̂2 or (CA239/NAT239). In a further aspect, the first score is calculated using the algorithm (CA157/CAvasp)/(NAT157/NATvasp) or (CA157/CAvasp), and the second score is calculated using the algorithm: SQRT(CA239/NAT239×CA157/NAT157)/(CAvasp/NATvasp)̂2 or (CA239/NAT239).

The first predetermined threshold, the second predetermined threshold, or both the first predetermined threshold and the second predetermined threshold, can have a sensitivity of at least 80%, of at least 85%, of at least 90%, of at least 95% or of at least 99%. The first predetermined threshold, the second predetermined threshold, or both the first predetermined threshold and the second predetermined threshold, can have a specificity of at least 40%, of at least 50%, of at least 60%, of at least 70%, of at least 75%, of at least 80%, of at least 85%, of at least 90%, of at least 95% or of at least 99%. The first predetermined threshold can have a negative predictive value of at least 85%, of at least 90%, of at least 95% or of at least 99%. The second predetermined threshold has a positive predictive value of at least 50%, of at least 60%, of at least 70%, of at least 75%, of at least 80%, of at least 85%, of at least 90%, of at least 95% or of at least 99%.

In some aspects, the subject was previously treated for a proliferation disorder. In a preferred aspect that proliferation disorder is cancer. The previous treatment can be surgery, chemotherapy, immunotherapy, radiotherapy, or a combination thereof. The subject can present with disease symptoms or be asymptomatic.

The clinical assessment is can be risk of recurrence of the proliferation disorder, preferably risk of recurrence of cancer. A test negative subject excluded from treatment has a low risk of recurrence of a proliferation disorder, preferably low risk of recurrence of cancer. A test positive subject recommended to receive treatment has a high risk of recurrence of a proliferation disorder, preferably high risk of recurrence of cancer. The recommendation of treatment can include chemotherapy, immunotherapy, radiotherapy, or a combination thereof.

The present disclosure also provides a method for providing a clinical assessment of a subject in need thereof including a) measuring the amount of at least one first biomarker in at least one first sample and the amount of at least one second biomarker in at least one second sample from the subject to generate a first score, wherein one of the at least first sample and at least second sample is tumor tissue and at least one of the at least first sample and at least second sample is normal tissue adjacent to the tumor tissue; b) comparing the first score to a first predetermined threshold to determine if the subject is test positive or test negative, wherein the predetermined threshold has a negative predictive value of at least 80%; c) providing a clinical assessment, wherein if the subject is determined to be test negative, the clinical assessment comprises recommending the subject is excluded from treatment; d) measuring the amount of at least one third biomarker in at least one third sample and the amount of at least one fourth biomarker in at least one fourth sample from a subject determined to be test positive in step (b) to generate a second score, wherein one of the at least third sample and at least fourth sample is tumor tissue and at least one of the at least third sample and at least fourth sample is normal tissue adjacent to the tumor tissue; e) comparing the second score to a second predetermined threshold to determine if the subject is test positive for the second predetermined threshold, wherein the predetermined threshold has a positive predictive value of at least 40%; and f) providing a clinical assessment, wherein if the subject is determined to be test positive in step (e), the clinical assessment comprises recommending the subject receive treatment.

The at least one first biomarker and the at least one second biomarker can be the same or can be different. The at least one third biomarker and the at least one fourth biomarker can be the same or can be different. The at least one first biomarker, at least one second biomarker, at least one third biomarker and at least one fourth biomarker can be an amino acid molecule, a protein, a polypeptide, a nucleic acid molecule, DNA, RNA, a lipid, a carbohydrate, or a combination thereof.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents, and other references mentioned herein are incorporated by reference. The references cited herein are not admitted to be prior art to the claimed disclosure. In the case of conflict, the present specification, including definitions, will control. In addition, the materials, methods, and examples are illustrative only and are not intended to be limiting.

Other features and advantages of the disclosure will be apparent from the following detailed description and claims.

### BRIEF DESCRIPTION OF THE DRAWINGS

FIG. 1A shows contingency tables generated from a single-tier test and a two tier-test with an incidence of 5%, and specificity and sensitivity of 80%. 25% of non-recurrent patients were excluded from the second tier.

FIG. 1B shows contingency tables generated from a single-tier test and a two tier-test with an incidence of 20%, and specificity and sensitivity of 80%. 25% of non-recurrent patients were excluded from the second tier.

FIG. 1C shows contingency tables generated from a single-tier test and a two tier-test with an incidence of 35%, and specificity and sensitivity of 80%. 25% of non-recurrent patients were excluded from the second tier.

FIG. 1D shows contingency tables generated from a single-tier test and a two tier-test with an incidence of 20%, and specificity and sensitivity of 80%. 25% of non-recurrent patients were excluded from the second tier.

FIG. 2A shows contingency tables generated from a single-tier test and a two tier-test with an incidence of 5%, and specificity and sensitivity of 80%. 50% of non-recurrent patients were excluded from the second tier.

FIG. 2B shows contingency tables generated from a single-tier test and a two tier-test with an incidence of 20%, and specificity and sensitivity of 80%. 50% of non-recurrent patients were excluded from the second tier.

FIG. 2C shows contingency tables generated from a single-tier test and a two tier-test with an incidence of 35%, and specificity and sensitivity of 80%. 50% of non-recurrent patients were excluded from the second tier.

FIG. 2D shows contingency tables generated from a single-tier test and a two tier-test with an incidence of 50%, and specificity and sensitivity of 80%. 50% of non-recurrent patients were excluded from the second tier.

FIG. 3A shows contingency tables generated from a single-tier test and a two tier-test with an incidence of 5%, and specificity and sensitivity of 80%. 75% of non-recurrent patients were excluded from the second tier.

FIG. 3B shows contingency tables generated from a single-tier test and a two tier-test with an incidence of 20%, and specificity and sensitivity of 80%. 75% of non-recurrent patients were excluded from the second tier.

FIG. 3C shows contingency tables generated from a single-tier test and a two tier-test with an incidence of 35%, and specificity and sensitivity of 80%. 75% of non-recurrent patients were excluded from the second tier.

FIG. 3D shows contingency tables generated from a single-tier test and a two tier-test with an incidence of 50%, and specificity and sensitivity of 80%. 75% of non-recurrent patients were excluded from the second tier.

FIG. 4 shows contingency tables generated from a single-tier test and a two tier-test with a 20% incidence of recurrence, using a threshold generated by ROC analysis.

FIG. 5 shows contingency tables generated a patient population with 50% incidence using data from VASP biomarkers and a threshold generated by ROC analysis.

FIG. 6A shows images (magnification, 10×) of primary tumors and matched NAT from 119 CRC patients with stage 0-II (TMA-1 on left) and stage III-IV (TMA 2 on right) disease.

FIG. 6B shows representative images (magnification, 20×) of primary and matched NAT mounted as whole-tissue sections. Tissues were stained (in brown) with the specific primary antibody for VASP, pSer157-VASP or pSer239-VASP and hematoxylin (blue, for nuclei).

FIG. 7A shows scatter plots of IHC scoring for VASP (n=93), pSer157-VASP (n=94) and pSer239-VASP (n=94) and include individual values of matched tumor and NAT pairs. Mean values with standard deviation are shown on the scatterplots. **** indicates p<0.0001 by two-tailed, paired t-test.

FIG. 7B shows boxplots of IHC scoring for VASP-normalized ratios of pSer157-VASP (left panel) in carcinomas in-situ (n=9) and matched NAT (n=11), and adenocarcinoma tumors (n=101) and matched NAT (n=93), or pSer239-VASP (right panel) in pre-invasive tumor (n=9) and matched NAT (n=11), and invasive tumor (n=100) and matched NAT (n=95). Box and whisker plots indicate median values and include 25^{th}-75^{th} percentiles. For preinvasive to invasive comparisons, ****, p<0.0001; **, p=0.003 by two-tailed, unpaired t-test. For tumor to NAT comparisons only tissues with matched tumor and NAT were included in the analysis; ****, p<0.0001; **, p=0.001 by two-tailed, paired t-test.

FIG. 7C shows boxplots of staining intensity ratios of VASP-normalized pSer157-VASP (left panel) or pSer239-VASP (right panel) in tumors over matched NAT (TNM stages I-III). Box and whisker plots indicate median values and include 25^{th}-75^{th} percentiles. N of tissues quantified were: pSer157-VASP/VASP (Tumor/NAT), 46 (N0) and 27 (N+); pSer239-VASP/VASP (Tumor/NAT), 44 (N0) and 27 (N+). **, p<0.002 by two-tailed, unpaired t-test.

FIG. 8. Shows scatterplots of semi-quantitatively and independently quantified staining intensities of VASP (left panel), pSer157-VASP (middle panel) and pSer239-VASP (right panel) using the H-score system (as described in Methods). The individual H-scores of identical IHC-stained tissues from two clinical pathologists who did not have knowledge of clinical outcomes or each other's H-score evaluations, were compared with the Spearman Correlation test. Significant correlations between the two pathologists' scores were obtained (VASP, p=0.045; pSer157-VASP, p<0.0003; pSer157-VASP, p<0.0001).

FIG. 9A shows boxplots of IHC scoring for individual biomarkers (left panel) or VASP-normalized ratios of pSer157-VASP and pSer239-VASP (right panel). ****, p<0.0001; **, p=0.003; *, p<0.05 by two-tailed, paired t-test.

FIG. 9B shows a graph of hazard ratios for recurrence and 95% Confidence Intervals associated with traditional pathological parameters and VASP biomarkers. **, p=0.002; *, p<0.05 by log-rank (Mantel-Cox) test.

FIG. 9C shows a schematic diagram of two-tiered testing model (upper left panel) and Kaplan Meier survival curves. A Kaplan-Meier survival curve associated with the Tier-1 prognostic biomarker VASP-normalized pSer157-VASP (upper right panel) is shown. A Kaplan-Meier survival curve of the Tier-2 prognostic biomarker pSer239-VASP tumor/NAT, before (lower left panel) and after (lower right panel) patients' exclusion by Tier-1 testing is shown.

FIG. 10A shows Receiver Operatic Characteristic curves (upper panels) and Kaplan-Meier survival curves (lower panels) for the cases in the whole-section study population as assessed by VASP-normalized pSer157-VASP and pSer239-VASP.

FIG. 10B shows Receiver Operatic Characteristic curves (upper panels) and Kaplan-Meier survival curves (lower panels) for relative tumor/NAT ratios of each VASP biomarker.

FIG. 10C shows a Receiver Operatic Characteristic curve (upper panel) and a Kaplan-Meier survival curve (lower panel) for an algorithm (detailed in Methods) integrating multiple VASP biomarkers into a single index score.

### DETAILED DESCRIPTION OF THE INVENTION

The present disclosure provides biomarkers and methods for assessing the clinical status of a patient. In particular, the invention provides methods for identifying the presence of or likelihood of disease or disease recurrence. In practice, methods of the invention provide the ability to screen patients into one of two distinct clinical categories. Based upon measurement of clinically-relevant biomarkers in a sample obtained from a patient, the invention allows the unambiguous identification of patients who are not at risk for or do not have the relevant disease or the unambiguous identification of patients at increased risk or who have the disease. Use of the invention maximizes the number of patients who will receive accelerated intervention or monitoring and minimizes those patients who will receive unnecessary standard of care or accelerated intervention or monitoring.

Methods of the invention are particularly useful in the clinical assessment of disease recurrence. Practice of the invention allows the unambiguous identification of patients who are not at risk for disease recurrence or who do not have recurrent disease, and those who are at heightened risk of recurrence or who have recurrent disease. Thus, practice of the invention allows a clinician to differentially stratify patients in order to reduce or eliminate treatment for an entire group of patients. The invention also provides means to identify those patients requiring increased monitoring and/or intervention. Practice of the invention allows a clinician to eliminate patients from further diagnostic or therapeutic intervention who are have no to low risk of disease and to increase intervention for patients who are high risk.

In one aspect, the present disclosure provides a method for providing a clinical assessment of a subject in need thereof including a) measuring the amount of at least one first biomarker in at least one first sample from the subject to generate a first score; b) comparing the first score to a first predetermined threshold to determine if the subject is test positive or test negative, wherein the predetermined threshold has a negative predictive value of at least 80%; c) providing a clinical assessment, wherein if the subject is determined to be test negative, the clinical assessment comprises recommending the subject is excluded from treatment; d) measuring the amount of at least one second biomarker in at least one second sample from a subject determined to be test positive in step (b) to generate a second score; e) comparing the second score to a second predetermined threshold to determine if the subject is test positive for the second predetermined threshold, wherein the predetermined threshold has a positive predictive value of at least 40%; and f) providing a clinical assessment, wherein if the subject is determined to be test positive in step (e), the clinical assessment comprises recommending the subject receive treatment.

In another aspect, the present disclosure also provides a method for providing a clinical assessment of a subject in need thereof including a) measuring the amount of at least one first biomarker in at least one first sample and the amount of at least one second biomarker in at least one second sample from the subject to generate a first score, wherein one of the at least first sample and at least second sample is tumor tissue and at least one of the at least first sample and at least second sample is normal tissue adjacent to the tumor tissue; b) comparing the first score to a first predetermined threshold to determine if the subject is test positive or test negative, wherein the predetermined threshold has a negative predictive value of at least 80%; c) providing a clinical assessment, wherein if the subject is determined to be test negative, the clinical assessment comprises recommending the subject is excluded from treatment; d) measuring the amount of at least one third biomarker in at least one third sample and the amount of at least one fourth biomarker in at least one fourth sample from a subject determined to be test positive in step (b) to generate a second score, wherein one of the at least third sample and at least fourth sample is tumor tissue and at least one of the at least third sample and at least fourth sample is normal tissue adjacent to the tumor tissue; e) comparing the second score to a second predetermined threshold to determine if the subject is test positive for the second predetermined threshold, wherein the predetermined threshold has a positive predictive value of at least 40%; and f) providing a clinical assessment, wherein if the subject is determined to be test positive in step (e), the clinical assessment comprises recommending the subject receive treatment.

The term biomarker, or biological marker, as used herein refers to a measurable indicator of some biological state or condition. For example, a measurable substance in a subject whose presence, absence and/or variation of amount (e.g. expression) is indicative of some phenomenon, such as a disease, disorder or condition.

A biomarker for use in the present disclosure can be any biological molecule, including but not limited to, an amino acid molecule, a protein, a polypeptide, a nucleic acid molecule, DNA, RNA, a lipid, a carbohydrate, a sugar, a glycan, or a combination thereof.

More specific examples of biomarkers to be utilized with the present disclosure include, but are not limited to, hormones (e.g., antidiuretic hormone (ADH), Adrenocorticotrophic hormone (ACTH), growth hormone(GH), follicle stimulating hormone (FSH), luteinizing hormone (LH), estrogen (estradiol, estrone, estriol), progesterone, testosterone, dihydrotestosterone (DHT), inhibin, somatotropin, dehydroepiandrostenedione (DHEA), somatostatin, glucagon, insulin, thyrotropin, thyroid stimulating hormone (TSH), thyroxin, parathyroid hormone, corticotropin, cortisol, corticosteron, aldosterone, epinephrine, norepinephrine, prolactin, vasopressin, oxytocin, melanocyte stimulating hormone (MSH)), growth factors (e.g., granulocyte-colony stimulating factor (G-CSF), granulocyte-macrophage colony stimulating factor (GM-CSF), nerve growth factor (NGF), neurotrophins, platelet-derived growth factor (PDGF), erythropeitin (EPO), thrmobopoeitin (TPO), myostatin (GDF-8), growth differentiation factor (GDF-9), basic fibroblast growth factor (bFGF or FGF2), acidic fibroblast growth factor, epidermal growth factor (EGF), hepatocyte growth factor (HGF), human stem cell factor (SCF), tumor necrosis factor (TNF), tumor necrosis factor-β (TNF-β), tumor necrosis factor-α (TNF-α), vascular endothelial growth factor (VEGF), transforming growth factor-β (TGF-β), transforming growth factor-α (TGF-α), insulin-like growth factor-I (IGF-II), insulin-like growth factor-II (IGF-II), and colony stimulating factor (CSF)), cytokines (e.g., IL-1α, IL-1β, IL-2, IL-3, IL-4, IL-5, IL-6, IL-7, IL-8, IL-9, IL-10, IL-11, IL-12, IL-13, IFN-α, IFN-β, and IFN-γ), proteins (e.g., Matrix metalloproteinases (MMPs) such as MMP2, MMP9, neutrophil gelatinase-associated lipocalin (NGAL), MMP/NGAL complex, thymosin β15, thymosin β16, collagen like gene (CLG) product, prohibitin, glutathione-S-transferase, beta-5-tubulin, ubiquitin, tropomyosin, Cyr61, cystatin B, chaperonin 10, profilin, Alpha-fetoprotein, Carcinoembryonic antigen, Epidermal growth factor receptor, Kallikrein 3 (prostate specific antigen), Vascular endothelial growth factor A, VEGF, Albumin, CA 125, Calcitonin, Chromogranin A (parathyroid secretory protein 1), Corticotropin-lipotropin contains ACTH, Estrogen receptor 1, Gastrin, Progesterone receptor, Prolactin, S100 alpha chain, Somatostatin, Thyroglobulin, V-erb-b2, Her2/neu, Antigen identified by monoclonal antibody Ki-67, B-cell CLUlymphoma 2, BCL2-associated X protein, Beta-2-microglobulin, Breast cancer 1 early onset, BRCA1, CA 15.3, CA 19.9, Cadherin 1 type 1 E-cadherin (epithelial), Caspase 3, CD44 antigen, Cellular tumor antigen p53, Coagulation factor II, prothrombin, Colony stimulating factor 2 (granulocyte-macrophage), Colony stimulating factor 3 (granulocyte), C-reactive protein, Cyclin D1, Cyclin-dependent kinase inhibitor 1, p21, Erythropoietin, Fibrinogen alpha/alpha-E chain, Follicle-stimulating hormone, Gamma enolase, Insulin, Interferon gamma, Interleukin 2, Interleukin 6, k-ras, k-ras2, Neprilysin, CD10, Transferrin, Trypsin, Tumor necrosis factor (TNF-alpha), Tumor necrosis factor receptor superfamily member 6, fas, Von Willebrand Factor, Chemokine, Chitinase-3 like protein 1, YKL-40, Choriogonadotropin beta chain, Colony stimulating factor 1 (macrophage), Haptoglobin-1, Hepatocyte growth factor, Inhibin, Interferon-alpha/beta receptor alpha chain, Interferon-alpha/beta receptor beta chain, Kallikrein 10, Kallikrein 11, Kallikrein 6, Matrix metalloproteinase 3, ADAM-12, Small inducible cytokine A21 (CCL21) soluble IL-2R alpha, Somatotropin growth factor, growth hormone, Breast cancer 2 early onset, BRCA2, Catenin Beta 1, Cathepsin D, CD15, Desmin, DNA-(apurinic or apyrimidinic site) lyase, APEX, Lutropin beta chain, Luteinizing hormone, Parathyroid Hormone, Proliferating cell nuclear antigen, Tumor necrosis factor ligand superfamily member 8 (CD30 ligand), V-myc myelocytomatosis viral oncogene homolog (avian), Tumor necrosis factor ligand superfamily member 8 (CD30), 17beta-Hydroxysteroid dehydrogenase type 1 (17HSD1), Acid phosphatase prostate, Adrenomedullin, Aldolase A, bone-specific Alkaline phosphatase, Alkaline phosphatase, placental type, Alpha-1-acid glycoprotein 1, orosomucoid, Alpha-1-antitrypsin, alpha-2-H S-glycoprotein, Alpha-2-macroglobulin, Alpha-lactalbumin, Angiogenin ribonuclease RNase A family 5, Angiopoietin 1, Angiopoietin 2, Antileukoproteinase 1, SLPI, Apolipoprotein A1, Apolipoprotein A-II, Apolipoprotein C-1, Apolipoprotein C-III, Bone sialoprotein II, Brain-derived neurotrophic factor, Breast cancer metastasis-suppressor 1, CA 27.29, CA 72-4, Cathepsin B, CC chemokine 4, HCC-4, CD44 variant V5 soluble, Ceruloplasmin, Cervical cancer 1 protooncogene protein p40, Chemokine (C-C motif) ligand 4 Small inducible cytokine A4 (CCL4), MIP-1-beta, Claudin-3, Claudin-4, Clusterin, Coagulation factor III, Coagulation factor XIII A chain, Coagulation factor XIII B chain, Collagen I c-terminal telopeptide, Complement component 3, Complement component 4, Complement component 7, Complement factor H related protein, Cyclin-dependent kinase 6, Cyclooxygenase-2, Cystatin A, Cystatin B, Cystatin C, Cytokeratin 8, Diazepam binding inhibitor, Endoglin, Endothelin 1, Epidermal growth factor, E-selectin, Ferritin H, Fibroblast growth factor 2 (basic), Fibronectin 1, Flt-3 ligand, Fms-related tyrosine kinase 1, VEGFRI, Folli statin, Fructose-bisphosphate aldolase B, Fructose-bisphosphate aldolase C, Geminin, Glucose-6-phosphate isomerase, Glypican-3, n-terminal, Growth arrest and DNA-damage-inducible alpha, Immunosuppressive acidic protein, Insulin-like growth factor 1 (somatomedin C), Insulin-like growth factor 2 (somatomedin A), Insulin-like growth factor binding protein 1, Insulin-like growth factor binding protein 2, Insulin-like growth factor binding protein 3, Intercellular Adhesion Molecule 1, Interferon alpha 1, Interleukin 1 alpha, Interleukin 1 beta, Interleukin 10, Interleukin 12A, Interleukin 16, Interleukin 5, Interleukin 6 receptor, Interleukin 6 signal transducer, Interleukin 7, Interleukin 8, Interleukin 9, Interleukin-1 receptor antagonist protein, IRAP, Kallikrein 14 (hK14), Kallikrein 2 prostatic, Kallikrein 5, Kallikrein 7, Kallikrein 8, Kallikrein 18, Kallikrein 8, Keratin 18, Keratin, type I cytoskeletal 19, cytokeratin 19, Kit ligand, Lactotransferrin, Leptin, L-selectin, Luteinizing hormone-releasing hormone receptor, Mac-2 Binding Protein 90K, Mammaglobin B, Mammary Serum, Antigen, Mast/stem cell growth factor receptor, Melanoma-inhibiting activity, Membrane cofactor protein, CD46 antigen, Mesothelin, Midkine, MK-1 protein, Ep-CAM, Myoblast determination protein 1, Nerve growth factor beta, Netrin-1, Neuroendocrine secretory protein-55, Neutrophil defensin 1, Neutrophil defensin 3, Nm23-H 1, OVX1, OX40, p65 oncofetal protein, Pancreatic secretory trypsin inhibitor, TATI, Parathyroid hormone-related protein, Pcaf, P300/CBP-associated factor, Pepsinogen-1, Placental specific tissue protein 12 Plasma retinol-binding protein, Plasminogen (Contains Angiostatin), Platelet endothelial cell adhesion molecule, PECAM-1, Platelet factor 4, Platelet-derived growth factor beta polypeptide, Platelet-derived growth factor receptor alpha polypeptide, Pregnancy zone protein, Pregnancy-associated plasma protein-A, Prostate secretory protein PSP94, P-selectin, PSP94 binding protein, Pyruvate kinase, isozymes M1/M2, Riboflavin carrier protein, 100 beta chain, Secreted phosphoprotein 1, osteopontin, Serine (or cysteine) proteinase inhibitor clade B, maspin, Serine (or cysteine) proteinase inhibitor clade E, PAI-1, Serum amyloid alpha-1, Serum paraoxonase/arylesterase 1, Small inducible cytokine A14 CCL14, Small inducible cytokine A18(CCL18), MIP-4, Small inducible cytokine A2(CCL2), Small inducible cytokine A3(CCL3), Macrophage inflammatory protein 1-alpha, Small inducible cytokine B5(CXCLS), Squamous cell carcinoma antigen 1, Squamous cell carcinoma antigen 2, Survivin, Syndecan-1, synuclein-gamma, TEK tyrosine kinase endothelial, Tie-2, Tenascin, Tetranectin, TGF-beta receptor type III, Thiredoxin reductase 1, Thrombopoietin, Thrombopoietin 1, Thymidin kinase, Tissue inhibitor of metalloproteinasel, Tissue inhibitor of metalloproteinase2, Tissue-type plasminogen activator, tPA, Transferrin receptor (p90 CD71), Transforming growth factor alpha, Transforming growth factor beta 1, transthyretin, Tropomyosin 1 alpha chain (Alpha-tropomyosin), Tumor necrosis factor (ligand) superfamily member 5, CD154, Tumor necrosis factor (ligand) superfamily member 6, Fas ligand, Tumor necrosis factor ligand superfamily member 13B, TALL-1, Tumor necrosis factor receptor superfamily member 11B, osteoprotegerin, Tumor necrosis factor receptor superfamily member 1A p60 TNF-RI p55 CD120a, TNFR1, Tumor necrosis factor receptor superfamily member 1B, TNFR2, Urokinase plasminogen activator surface receptor, U-PAR, Vascular cell adhesion molecule 1, Vascular endothelial growth factor receptor 2, Vasoactive intestinal peptide, VEGF (165)b, Vitamin K dependent protein C, Vitronectin, and X box binding protein-1), antibodies, APC, DCC, TP53, PRC1, NUSAP1, CAPZ, PFKP, EVER1, FLT1, ESPL1, AKAP2, CDC45L, RAMP, SYNGR2, NDRG1, ZNF533, Vasodilator-stimulated phosphoprotein (VASP), or any combination thereof.

In a preferred aspect, the biomarker is Vasodilator-stimulated phosphoprotein (VASP). In one aspect, a VASP biomarker of the present invention comprises the nucleic acid sequence from NCBI (NM_003370.3) as shown in SEQ ID NO:1 (start (atg) and stop (tga) codons are bolded and underlined):

| | |
|---|---|
| 1 | tgtgggtgcg gggagtggaa ttttggaacg aaatgtaacg aagagaagta cagtagtaag |
| 61 | agtaacactg tagccgccac cggcaagggg tgcgcgctgg ggagcggacg ctgcatcccc |
| 121 | tttctgctgc aggaacctct catcagaccg cctgagggaa gcggcgcccg gagacccgcc |
| 181 | ccggcccggt ccacattctc cccaggaagc cggactctat ggggcgggac cctgggggag |
| 241 | cctgagccga gcccggagcc agccccgaac ccctgaacct ccagccaggg gcgccccggg |
| 301 | agcagccagc ccgtgggcga gccgcccgcc cgccgagcag ccatgagcga gacggtcatc |
| 361 | tgttccagcc gggccactgt gatgctttat gatgatggca acaagcgatg gctccctgct |
| 421 | ggcacgggtc cccaggcctt cagccgcgtc cagatctacc acaaccccac ggccaattcc |
| 481 | tttcgcgtcg tgggccggaa gatgcagccc gaccagcagg tggtcatcaa ctgtgccatc |
| 541 | gtccggggtg tcaagtataa ccaggccacc cccaacttcc atcagtggcg cgacgctcgc |
| 601 | caggtctggg gcctcaactt cggcagcaag gaggatgcgg cccagtttgc cgccggcatg |
| 661 | gccagtgccc tagaggcgtt ggaaggaggt gggccccctc cacccccagc acttcccacc |
| 721 | tggtcggtcc cgaacggccc ctccccggag gaggtggagc agcagaaaag gcagcagccc |
| 781 | ggcccgtcgg agcacataga gcgccgggtc tccaatgcag gaggcccacc tgctcccccc |
| 841 | gctgggggtc cacccccacc accaggacct ccccctcctc caggtccccc cccaccccca |
| 901 | ggtttgcccc cttcgggggt cccagctgca gcgcacggag cagggggagg accaccccct |
| 961 | gcaccccctc tcccggcagc acagggccct ggtggtgggg gagctggggc cccaggcctg |
| 1021 | gccgcagcta ttgctggagc caaactcagg aaagtcagca agcaggagga ggcctcaggg |
| 1081 | gggcccacag cccccaaagc tgagagtggt cgaagcggag gtgggggact catggaagag |
| 1141 | atgaacgcca tgctggcccg gagaaggaaa gccacgcaag ttggggagaa aacccccaag |
| 1201 | gatgaatctg ccaatcagga ggagccagag gccagagtcc cggcccagag tgaatctgtg |
| 1261 | cggagaccct gggagaagaa cagcacaacc ttgccaagga tgaagtcgtc ttcttcggtg |
| 1321 | accacttccg agacccaacc ctgcacgccc agctccagtg attactcgga cctacagagg |
| 1381 | gtgaaacagg agcttctgga agaggtgaag aaggaattgc agaaagtgaa agaggaaatc |
| 1441 | attgaagcct tcgtccagga gctgaggaag cggggttctc cctgaccaca gggacccaga |
| 1501 | agacccgctt ctcctttccg cacacccggc ctgtcaccct gctttccctg cctctacttg |
| 1561 | acttggaatt ggctgaagac tacacaggaa tgcatcgttc ccactcccca tcccacttgg |
| 1621 | aaaactccaa gggggtgtgg cttccctgct cacacccaca ctggctgctg attggctggg |
| 1681 | gaggcccccg cccttttctc cctttggtcc ttcccctctg ccatcccctt ggggccggtc |
| 1741 | cctctgctgg ggatgcacca atgaacccca caggaagggg gaaggaagga gggaatttca |
| 1801 | cattcccttg ttctagattc actttaacgc ttaatgcctt caaagttttg gtttttttaa |
| 1861 | gaaaaaaaaa tatatatata tttgggtttt gggggaaaag ggaaattttt ttttctcttt |
| 1921 | ggttttgata aaatgggatg tgggagtttt taaatgctat agccctgggc ttgccccatt |
| 1981 | tggggcagct atttaagggg aggggatgtc tcaccgggct gggggtgaga tatcccccca |
| 2041 | ccccagggac tccccttccc tctggctcct tccccttttc tatgaggaaa taagatgctg |
| 2101 | taactttttg gaacctcagt tttttgattt tttatttggg taggttttgg ggtccaggcc |
| 2161 | atttttttta ccccttggag gaaataagat gagggagaaa ggagaagggg aggaaacttc |
| 2221 | tcccctccca ccttcacctt tagcttcttg aaaatgggcc cctgcagaat aaatctgcca |
| 2281 | gtttttataa aaaaaaaa |

In one aspect, a VASP biomarker of the present invention comprises the amino acid sequence from NCBI (NP_003361.1) as shown in SEQ ID NO:2:

| | |
|---|---|
| 1 | MSETVICSSR ATVMLYDDGN KRWLPAGTGP QAFSRVQIYH NPTANSFRVV GRKMQPDQQV |
| 61 | VINCAIVRGV KYNQATPNFH QWRDARQVWG LNFGSKEDAA QFAAGMASAL EALEGGGPPP |
| 121 | PPALPTWSVP NGPSPEEVEQ QKRQQPGPSE HIERRVSNAG GPPAPPAGGP PPPPGPPPPP |
| 181 | GPPPPPGLPP SGVPAAAHGA GGGPPPAPPL PAAQGPGGGG AGAPGLAAAI AGAKLRKVSK |
| 241 | QEEASGGPTA PKAESGRSGG GGLMEEMNAM LARRRKATQV GEKTPKDESA NQEEPEARVP |
| 301 | AQSESVRRPW EKNSTTLPRM KSSSSVTTSE TQPCTPSSSD YSDLQRVKQE LLEEVKKELQ |
| 361 | KVKEEIIEAF VQELRKRGSP |

In some aspects, a VASP protein biomarker is phosphorylated at amino acid residue 157 of SEQ ID NO:2 (VASP157 or 157), phosphorylated at amino acid residue 239 of SEQ ID NO:2 (VASP239 or 239), or phosphorylated at both amino acid residue 157 and amino acid residue 239 of SEQ ID NO:2 (VASP157/239 or 157/239). Residues 157 and 239 are bolded and underlines in SEQ ID NO:2 above.

In some aspects, VASP157, VASP239 or both VASP157 and VASP239 protein is measured in a tissue sample. In some aspects, total VASP protein in a tissue sample is measured (referred to as VASP or total VASP). In some aspects, the VASP biomarker is detected in tumor or cancerous tissue (CA) or normal tissue adjacent to tumor or cancerous tissue (normal adjacent tissue or NAT). VASP157 measured in tumor or cancerous tissues is termed CA157 herein. VASP239 measured in tumor or cancerous tissues is termed CA239 herein. Total VASP measured in tumor or cancerous tissues is termed CAvasp herein. VASP157 measured in normal adjacent tissue is termed NAT157 herein. VASP239 measured in normal adjacent tissue is termed NAT239 herein. Total VASP measured in normal adjacent tissue is termed NATvasp herein.

The at least one first biomarker and the at least one second biomarker can be the same or can be different. In some aspects of the disclosure, the at least one third biomarker and the at least one fourth biomarker can be the same or can be different. In some aspects of the disclosure, the at least first biomarker, at least second biomarker, at least third biomarker and at least fourth biomarker can be the same or different.

A different biomarker can be one that is completely distinct, structurally and or functionally (e.g., tumor necrosis factor (TNF) and Vasodilator-stimulated phosphoprotein (VASP)). A different biomarker can also be one that is the same biomarker but has undergone a mutation, e.g., a single nucleotide polymorphism (SNP). A different biomarker can also be one that is the same biomarker but with a chemical modification. For example, nucleic acid markers (e.g., DNA, RNA) may undergo epigenetic and chemical modifications, including, but not limited to, methylation, hypermethylation, and demethylation. For example, protein or polypeptide biomarkers may undergo chemical or posttranslational modifications (e.g., phosphorylation at serine, threonine, or tyrosine residues; each of these phosphorylated species may be a different biomarker). Protein and polypeptide biomarkers may also undergo other chemical or posttranslational modifications including, but not limited to, acetylation, ubiquitination, alkylation, glycosylation, hydroxylation, amidation, methylation, and oxidation, generating different or unique biomarkers for disease screening and treatment.

A “sample” as used herein can be any cell, tissue or bodily fluid. In non-limiting examples, the sample can be tumor tissue, normal tissue, normal tissue adjacent to a tumor, saliva, plasma, blood, serum, spinal fluid, lymphatic fluid, urine, or a combination thereof. In one aspect, the at least one first sample and the at least one second sample can be the same or can be different. The at least first sample or the at least second sample can be any cell, tissue or bodily fluid. For example the at least first sample or the at least second sample can be a tumor tissue, normal tissue, normal tissue adjacent to a tumor, saliva, plasma, blood, serum, spinal fluid, lymphatic fluid, urine, or a combination thereof. In some aspects, the at least first sample or the at least second sample is tumor tissue. In particular, the tumor tissue is an epithelial tumor tissue. In some aspects, the at least first sample or the at least second sample is a tumor tissue and normal tissue adjacent to that tumor tissue.

A score is a useful metric that may be generated for clinical assessment of any disease. A clinical assessment may also be called a test. A score may measure indicia of health or disease status of a subject. For example, a score may measure of at least one biomarker associated with health or disease status. A score may be set within any acceptable range. For example, a score within a 0 to 1 range. In some aspects, a first score, a second score, or both a first and second score, are calculated. In some aspects, the first score and second score are the same. In other aspects, the first score and the second score are different.

A score calculated from an algorithm. In some aspects, a first score is calculated from an algorithm. In some aspects, a second score is calculated from an algorithm. In some aspects, the same algorithm is used to calculate the first score and the second score. In other aspects, the algorithms used to calculate the first score and the second score are different. In various embodiments, the algorithm used is based on hazard ratio. In various embodiments, the algorithm used is based on negative predictive value (NPV). Without limitation, the algorithm may be based on relative risk, odds ratio, positive predictive value, logistic regression (e.g. logarithmic regression), linear regression, polynomial regression, logistic regression, multivariate linear regression, or Gaussian function. Other statistical measures that can be used in an algorithm are known in the art.

In one aspect, the algorithm that is used to calculate the score is selected from (CA157/CAvasp)/(NAT157/NATvasp); (CA157/CAvasp); SQRT(CA239/NAT239×CA157/NAT157)/(CAvasp/NATvasp)̂2 or (CA239/NAT239). In a further aspect, the first score is calculated using the algorithm (CA157/CAvasp)/(NAT157/NATvasp) or (CA157/CAvasp), and the second score is calculated using the algorithm: SQRT(CA239/NAT239×CA157/NAT157)/(CAvasp/NATvasp)̂2 or (CA239/NAT239).

In some aspects, a score is compared to a threshold. The threshold may be predetermined or calculated at the time of assessment. The threshold can be obtained from the literature, from known indications or can be derived empirically. In some aspects, a first score is compared to a first threshold. In some aspects, a second score is compared to a second threshold. In some aspects, the first threshold value is predetermined. In some aspects, the second threshold value is predetermined. In some aspects, the first predetermined threshold and the predetermined second threshold are the same. In other aspects, the first predetermined threshold and the predetermined second threshold are different.

The threshold can be calculated using an algorithm. In some aspects, the first predetermined threshold is calculated using an algorithm. In some aspects, the second predetermined threshold is calculated using an algorithm. In some aspects, both the first predetermined threshold and the second predetermined threshold are calculated using an algorithm.

In one aspect, the algorithm that is used to calculate the threshold is selected from (CA157/CAvasp)/(NAT157/NATvasp); (CA157/CAvasp); SQRT(CA239/NAT239×CA157/NAT157)/(CAvasp/NATvasp)̂2; or (CA239/NAT239). In a further aspect, the first predetermined threshold is calculated using the algorithm (CA157/CAvasp)/(NAT157/NATvasp) or (CA157/CAvasp), and the second predetermined threshold is calculated using the algorithm: SQRT(CA239/NAT239×CA157/NAT157)/(CAvasp/NATvasp)̂2 or (CA239/NAT239).

The threshold value may be optimized to discriminate between patient groups. For example, patients may be healthy or disease free; low-risk or high-risk, recurrent or non-recurrent for a given disease; etc. The threshold value may be optimized to maximize the number of patients who will receive a recommendation for treatment. In some embodiments, the threshold is optimized to classify a patient as test positive. In some embodiments, the threshold is optimized to classify a patient as test negative. In a preferred embodiment, an optimal threshold value is calculated by receiver operating characteristic (ROC) curve analysis.

As used herein, NPV is defined as the percentage of people who test negative that are actually negative. In a some aspects, a threshold has a negative predictive value of at least 80%, 80%, 85%, 90%, 91%, 92%, 93%, 94%, 95%, 96%, 97%, 98%, 99%, or 100%, inclusive of the endpoints. In a some aspects, the first predetermined threshold has a negative predictive value of at least 85%, 90%, 91%, 92%, 93%, 94%, 95%, 96%, 97%, 98%, 99%, or 100%, inclusive of the endpoints. In a some aspects, the second predetermined threshold has a negative predictive value of at least 80%, 85%, 90%, 91%, 92%, 93%, 94%, 95%, 96%, 97%, 98%, 99%, or 100%, inclusive of the endpoints.

As used herein, PPV is defined as the percentage of people who test positive that are actually positive. In a some aspects, a predetermined threshold has a PPV of at least 0%, 10%, 20%, 30%, 40%, 50%, 60%, 70%, 80%, 91%, 92%, 93%, 94%, 95%, 96%, 97%, 98%, 99%, or 100%, inclusive of the endpoints. In a some aspects, the first predetermined threshold has a PPV of at least 50%, 60%, 70%, 80%, 91%, 92%, 93%, 94%, 95%, 96%, 97%, 98%, 99%, or 100%, inclusive of the endpoints. In a some aspects, the second predetermined threshold has a PPV of at least 50%, 60%, 70%, 80%, 91%, 92%, 93%, 94%, 95%, 96%, 97%, 98%, 99%, or 100%, inclusive of the endpoints. It is known in the art that a predetermined threshold used in clinical assessment or test of a population of primarily healthy subjects may be associated with a low PPV. For example, a clinical assessment for measuring cervical cancer may have a predetermined threshold with a PPV of <10%.

In some aspect, the predetermined threshold is determined by sensitivity. Sensitivity is defined as the percentage of true positives assessed that are predicted by a clinical or assessment or a test to be positive. For example, a ROC curve provides the sensitivity of a test as a function of 1-specificity.

In some aspects, the predetermined threshold has a sensitivity of at least 80%, 85%, 90%, 91%, 92%, 93%, 94%, 95%, 96%, 97%, 98%, 99%, or 100%, inclusive of the endpoints. In some aspects, the first predetermined threshold has a sensitivity of at least 80%, 85%, 90%, 91%, 92%, 93%, 94%, 95%, 96%, 97%, 98%, 99%, or 100%, inclusive of the endpoints. In some aspects, the second predetermined threshold has a sensitivity of at least 80%, 85%, 90%, 91%, 92%, 93%, 94%, 95%, 96%, 97%, 98%, 99%, or 100%, inclusive of the endpoints. In some aspects, both the first predetermined threshold and second predetermined threshold has a sensitivity of at least 80%, 85%, 90%, 91%, 92%, 93%, 94%, 95%, 96%, 97%, 98%, 99%, or 100%, inclusive of the endpoints.

In some aspects, the predetermined threshold is determined by specificity. Specificity is defined as the percentage of true negatives assessed that are predicted by a test to be negative.

In some aspects, the predetermined threshold has a specificity of at least 40%, 50%, 60%, 70%, 75%, 80%, 85%, 90%, 91%, 92%, 93%, 94%, 95%, 96%, 97%, 98%, 99%, or 100%, inclusive of the endpoints. In some aspects, the first predetermined threshold has a specificity of at least 40%, 50%, 60%, 70%, 75%, 80%, 85%, 90%, 91%, 92%, 93%, 94%, 95%, 96%, 97%, 98%, 99%, or 100%, inclusive of the endpoints. In some aspects, the second predetermined threshold has a specificity of at least 40%, 50%, 60%, 70%, 75%, 80%, 85%, 90%, 91%, 92%, 93%, 94%, 95%, 96%, 97%, 98%, 99%, or 100%, inclusive of the endpoints. In some aspects, both the first predetermined threshold and the second predetermined threshold has a specificity of at least 40%, 50%, 60%, 70%, 75%, 80%, 85%, 90%, 91%, 92%, 93%, 94%, 95%, 96%, 97%, 98%, 99%, or 100%, inclusive of the endpoints.

In some aspects, the predetermined threshold has both a sensitivity and specificity of at least 80%, 85%, 90%, 91%, 92%, 93%, 94%, 95%, 96%, 97%, 98%, 99%, or 100%, inclusive of the endpoints. In some aspects, the first predetermined threshold has both a sensitivity and specificity of at least 80%, 85%, 90%, 91%, 92%, 93%, 94%, 95%, 96%, 97%, 98%, 99%, or 100%, inclusive of the endpoints. In some aspects, the second predetermined threshold has both a sensitivity and specificity of at least 80%, 85%, 90%, 91%, 92%, 93%, 94%, 95%, 96%, 97%, 98%, 99%, or 100%, inclusive of the endpoints. In some aspects, both the first and the second predetermined threshold have both a sensitivity and specificity of at least 80%, 85%, 90%, 91%, 92%, 93%, 94%, 95%, 96%, 97%, 98%, 99%, or 100%, inclusive of the endpoints.

The clinical assessment is can be risk of recurrence of a cell proliferation disorder. The cell proliferative disorder can be cancer. A test negative subject excluded from treatment has a low risk of recurrence of a cell proliferation disorder, preferably low risk of recurrence of cancer. A test positive subject recommended to receive treatment has a high risk of recurrence of a cell proliferation disorder, preferably high risk of recurrence of cancer.

As used herein, a “subject in need thereof” is a subject having a cell proliferative disorder, a subject previously treated for a cell proliferative disorder, or a subject having an increased risk of developing a cell proliferative disorder relative to the population at large. Preferably, a subject in need thereof has cancer, was previously treated for cancer, or is at increased risk of developing or having a recurrence of cancer. A “subject” includes a mammal. The mammal can be e.g., any mammal, e.g., a human, primate, bird, mouse, rat, fowl, dog, cat, cow, horse, goat, camel, sheep or a pig. Preferably, the mammal is a human. The term “subject” and the term “patient” are used interchangeably herein.

As used herein, the term “cell proliferative disorder” refers to conditions in which unregulated or abnormal growth, or both, of cells can lead to the development of an unwanted condition or disease, which may or may not be cancerous. Exemplary cell proliferative disorders encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. A cell proliferative disorder includes a precancer or a precancerous condition. A cell proliferative disorder includes cancer. The term “cancer” includes solid tumors, as well as, hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer.

Exemplary non-cancerous conditions or disorders include, but are not limited to, rheumatoid arthritis; inflammation; autoimmune disease; lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; psoriasis; eczema; ulcerative colitis; pancreatic fibrosis; hepatic fibrosis; acute and chronic renal disease; irritable bowel syndrome; pyresis; restenosis; cerebral malaria; stroke and ischemic injury; neural trauma; neurodegenerative disease or disorder; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; Multiple Sclerosis; lupus; fibromyalgia; AIDS and other viral diseases such as Herpes Zoster, Herpes Simplex I or II, influenza virus and cytomegalovirus; and diabetes mellitus.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, uringary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

A “cell proliferative disorder of the hematologic system” is a cell proliferative disorder involving cells of the hematologic system. A cell proliferative disorder of the hematologic system can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A cell proliferative disorder of the hematologic system can include hyperplasia, dysplasia, and metaplasia of cells of the hematologic system. A hematologic cancer can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

A “cell proliferative disorder of the colon” is a cell proliferative disorder involving cells of the colon. Preferably, the cell proliferative disorder of the colon is colon cancer. Colon cancer can include all forms of cancer of the colon. Colon cancer can include sporadic and hereditary colon cancers. Colon cancer can include malignant colon neoplasms, carcinoma in situ, typical carcinoid tumors, and atypical carcinoid tumors. Colon cancer can include adenocarcinoma, squamous cell carcinoma, and adenosquamous cell carcinoma. Colon cancer can be associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. Colon cancer can be caused by a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis.

Cell proliferative disorders of the colon can include all forms of cell proliferative disorders affecting colon cells. Cell proliferative disorders of the colon can include colon cancer, precancerous conditions of the colon, adenomatous polyps of the colon and metachronous lesions of the colon. A cell proliferative disorder of the colon can include adenoma. Cell proliferative disorders of the colon can be characterized by hyperplasia, metaplasia, and dysplasia of the colon. Prior colon diseases that may predispose individuals to development of cell proliferative disorders of the colon can include prior colon cancer. Current disease that may predispose individuals to development of cell proliferative disorders of the colon can include Crohn's disease and ulcerative colitis. A cell proliferative disorder of the colon can be associated with a mutation in a gene selected from the group consisting of p53, ras, FAP and DCC. An individual can have an elevated risk of developing a cell proliferative disorder of the colon due to the presence of a mutation in a gene selected from the group consisting of p53, ras, FAP and DCC.

A “cell proliferative disorder of the breast” is a cell proliferative disorder involving cells of the breast. Cell proliferative disorders of the breast can include all forms of cell proliferative disorders affecting breast cells. Cell proliferative disorders of the breast can include breast cancer, a precancer or precancerous condition of the breast, benign growths or lesions of the breast, and malignant growths or lesions of the breast, and metastatic lesions in tissue and organs in the body other than the breast. Cell proliferative disorders of the breast can include hyperplasia, metaplasia, and dysplasia of the breast.

A cell proliferative disorder of the breast can be a precancerous condition of the breast. A precancerous condition of the breast can include atypical hyperplasia of the breast, ductal carcinoma in situ (DCIS), intraductal carcinoma, lobular carcinoma in situ (LCIS), lobular neoplasia, and stage 0 or grade 0 growth or lesion of the breast (e.g., stage 0 or grade 0 breast cancer, or carcinoma in situ). A precancerous condition of the breast can be staged according to the TNM classification scheme as accepted by the American Joint Committee on Cancer (AJCC), where the primary tumor (T) has been assigned a stage of T0 or Tis; and where the regional lymph nodes (N) have been assigned a stage of N0; and where distant metastasis (M) has been assigned a stage of M0.

The cell proliferative disorder of the breast can be breast cancer. Breast cancer includes all forms of cancer of the breast. Breast cancer can include primary epithelial breast cancers. Breast cancer can include cancers in which the breast is involved by other tumors such as lymphoma, sarcoma or melanoma. Breast cancer can include carcinoma of the breast, ductal carcinoma of the breast, lobular carcinoma of the breast, undifferentiated carcinoma of the breast, cystosarcoma phyllodes of the breast, angiosarcoma of the breast, and primary lymphoma of the breast. Breast cancer can include Stage I, II, IIIA, IIIB, IIIC and IV breast cancer. Ductal carcinoma of the breast can include invasive carcinoma, invasive carcinoma in situ with predominant intraductal component, inflammatory breast cancer, and a ductal carcinoma of the breast with a histologic type selected from the group consisting of comedo, mucinous (colloid), medullary, medullary with lymphcytic infiltrate, papillary, scirrhous, and tubular. Lobular carcinoma of the breast can include invasive lobular carcinoma with predominant in situ component, invasive lobular carcinoma, and infiltrating lobular carcinoma. Breast cancer can include Paget's disease, Paget's disease with intraductal carcinoma, and Paget's disease with invasive ductal carcinoma. Breast cancer can include breast neoplasms having histologic and ultrastructual heterogeneity (e.g., mixed cell types).

A “cell proliferative disorder of the lung” is a cell proliferative disorder involving cells of the lung. Cell proliferative disorders of the lung can include all forms of cell proliferative disorders affecting lung cells. Cell proliferative disorders of the lung can include lung cancer, a precancer or precancerous condition of the lung, benign growths or lesions of the lung, and malignant growths or lesions of the lung, and metastatic lesions in tissue and organs in the body other than the lung. Lung cancer can include all forms of cancer of the lung. Lung cancer can include malignant lung neoplasms, carcinoma in situ, typical carcinoid tumors, and atypical carcinoid tumors. Lung cancer can include small cell lung cancer (“SCLC”), non-small cell lung cancer (“NSCLC”), squamous cell carcinoma, adenocarcinoma, small cell carcinoma, large cell carcinoma, adenosquamous cell carcinoma, and mesothelioma. Lung cancer can include “scar carcinoma”, bronchioalveolar carcinoma, giant cell carcinoma, spindle cell carcinoma, and large cell neuroendocrine carcinoma. Lung cancer can include lung neoplasms having histologic and ultrastructual heterogeneity (e.g., mixed cell types).

Cell proliferative disorders of the lung can include all forms of cell proliferative disorders affecting lung cells. Cell proliferative disorders of the lung can include lung cancer, precancerous conditions of the lung. Cell proliferative disorders of the lung can include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

As used herein, a “normal cell or normal tissue” is a cell or tissue that cannot be classified as part of a “cell proliferative disorder”. A normal cell or tissue lacks unregulated or abnormal growth, or both, that can lead to the development of an unwanted condition or disease. Preferably, a normal cell possesses normally functioning cell cycle checkpoint control mechanisms.

As used herein, a “normal tissue adjacent to tumor tissue” or “NAT” is a cell or tissue that cannot be classified as part of a “cell proliferative disorder” but that is next to, adjacent to or contacts a tissue deemed to part of a “cell proliferative disorder” in a subject.

The subject in need thereof can present with a sign or a symptom of the disease or be asymptomatic. As used herein the term “symptom” is defined as an indication of disease, illness, injury, or that something is not right in the body. Symptoms are felt or noticed by the individual experiencing the symptom, but may not easily be noticed by others. Others are defined as non-health-care professionals. As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. If the cancer is in a critical area, such as certain parts of the brain, even the smallest tumor can cause early symptoms. But sometimes cancers start in places where it does not cause any symptoms until the cancer has grown quite large. Pancreas cancers, for example, do not usually grow large enough to be felt from the outside of the body. Some pancreatic cancers do not cause symptoms until they begin to grow around nearby nerves (this causes a backache). Others grow around the bile duct, which blocks the flow of bile and leads to a yellowing of the skin known as jaundice. By the time a pancreatic cancer causes these signs or symptoms, it has usually reached an advanced stage.

A cancer may also cause symptoms such as fever, fatigue, or weight loss. This may be because cancer cells use up much of the body's energy supply or release substances that change the body's metabolism. Or the cancer may cause the immune system to react in ways that produce these symptoms.

Sometimes, cancer cells release substances into the bloodstream that cause symptoms not usually thought to result from cancers. For example, some cancers of the pancreas can release substances which cause blood clots to develop in veins of the legs. Some lung cancers make hormone-like substances that affect blood calcium levels, affecting nerves and muscles and causing weakness and dizziness.

Cancer presents several general signs or symptoms that occur when a variety of subtypes of cancer cells are present. Most people with cancer will lose weight at some time with their disease. An unexplained (unintentional) weight loss of 10 pounds or more may be the first sign of cancer, particularly cancers of the pancreas, stomach, esophagus, or lung.

Fever is very common with cancer, but is more often seen in advanced disease. Almost all patients with cancer will have fever at some time, especially if the cancer or its treatment affects the immune system and makes it harder for the body to fight infection. Less often, fever may be an early sign of cancer, such as with leukemia or lymphoma.

Fatigue may be an important symptom as cancer progresses. It may happen early, though, in cancers such as with leukemia, or if the cancer is causing an ongoing loss of blood, as in some colon or stomach cancers.

Pain may be an early symptom with some cancers such as bone cancers or testicular cancer. But most often pain is a symptom of advanced disease.

Along with cancers of the skin (see next section), some internal cancers can cause skin signs that can be seen. These changes include the skin looking darker (hyperpigmentation), yellow (jaundice), or red (erythema); itching; or excessive hair growth.

Alternatively, or in addition, cancer subtypes present specific signs or symptoms. Changes in bowel habits or bladder function could indicate cancer. Long-term constipation, diarrhea, or a change in the size of the stool may be a sign of colon cancer. Pain with urination, blood in the urine, or a change in bladder function (such as more frequent or less frequent urination) could be related to bladder or prostate cancer.

Changes in skin condition or appearance of a new skin condition could indicate cancer. Skin cancers may bleed and look like sores that do not heal. A long-lasting sore in the mouth could be an oral cancer, especially in patients who smoke, chew tobacco, or frequently drink alcohol. Sores on the penis or vagina may either be signs of infection or an early cancer.

Unusual bleeding or discharge could indicate cancer. Unusual bleeding can happen in either early or advanced cancer. Blood in the sputum (phlegm) may be a sign of lung cancer. Blood in the stool (or a dark or black stool) could be a sign of colon or rectal cancer. Cancer of the cervix or the endometrium (lining of the uterus) can cause vaginal bleeding. Blood in the urine may be a sign of bladder or kidney cancer. A bloody discharge from the nipple may be a sign of breast cancer.

A thickening or lump in the breast or in other parts of the body could indicate the presence of a cancer. Many cancers can be felt through the skin, mostly in the breast, testicle, lymph nodes (glands), and the soft tissues of the body. A lump or thickening may be an early or late sign of cancer. Any lump or thickening could be indicative of cancer, especially if the formation is new or has grown in size.

Indigestion or trouble swallowing could indicate cancer. While these symptoms commonly have other causes, indigestion or swallowing problems may be a sign of cancer of the esophagus, stomach, or pharynx (throat).

Recent changes in a wart or mole could be indicative of cancer. Any wart, mole, or freckle that changes in color, size, or shape, or loses its definite borders indicates the potential development of cancer. For example, the skin lesion may be a melanoma.

A persistent cough or hoarseness could be indicative of cancer. A cough that does not go away may be a sign of lung cancer. Hoarseness can be a sign of cancer of the larynx (voice box) or thyroid.

While the signs and symptoms listed above are the more common ones seen with cancer, there are many others that are less common and are not listed here. However, all art-recognized signs and symptoms of cancer are contemplated and encompassed by the instant invention.

The methods of the present disclosure include a recommendation of treatment, and may further comprising administering a treatment to a subject to whom a recommendation of treatment was provided. The treatment can include chemotherapy, immunotherapy, radiotherapy, or a combination thereof.

As used herein, “treating” or “treat” describes the management and care of a patient for the purpose of combating a disease, condition, or disorder and includes the administration of chemotherapy, immunotherapy, radiotherapy, or a combination thereof, to alleviate the symptoms or complications of a disease, condition or disorder, or to eliminate the disease, condition or disorder.

As used herein, the term “alleviating” or “alleviate” is meant to describe a process by which the severity of a sign or symptom of a disorder is decreased. Importantly, a sign or symptom can be alleviated without being eliminated.

A chemotherapeutic agent (also referred to as an anti-neoplastic agent or anti-proliferative agent) can be an alkylating agent; an antibiotic; an anti-metabolite; a detoxifying agent; an interferon; a polyclonal or monoclonal antibody; an EGFR inhibitor; a HER2 inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase inhibitor), a cytidine analogue drug or any chemotherapeutic, anti-neoplastic or anti-proliferative agent listed in www.cancer.org/docroot/cdg/cdg_0.asp.

Exemplary alkylating agents include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).

Exemplary antibiotics include, but are not limited to, doxorubicin (Adriamycin); doxorubicin liposomal (Doxil); mitoxantrone (Novantrone); bleomycin (Blenoxane); daunorubicin (Cerubidine); daunorubicin liposomal (DaunoXome); dactinomycin (Cosmegen); epirubicin (Ellence); idarubicin (Idamycin); plicamycin (Mithracin); mitomycin (Mutamycin); pentostatin (Nipent); or valrubicin (Valstar).

Exemplary anti-metabolites include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS).

Exemplary detoxifying agents include, but are not limited to, amifostine (Ethyol) or mesna (Mesnex).

Exemplary interferons include, but are not limited to, interferon alfa-2b (Intron A) or interferon alfa-2a (Roferon-A).

Exemplary polyclonal or monoclonal antibodies include, but are not limited to, trastuzumab (Herceptin); ofatumumab (Arzerra); bevacizumab (Avastin); rituximab (Rituxan); cetuximab (Erbitux); panitumumab (Vectibix); tositumomaModine¹³¹ tositumomab (Bexxar); alemtuzumab (Campath); ibritumomab (Zevalin; In-111; Y-90 Zevalin); gemtuzumab (Mylotarg); eculizumab (Soliris) ordenosumab.

Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (CI-1033); matuzumab (Emd7200) or EKB-569.

Exemplary HER2 inhibitors include, but are not limited to, trastuzumab (Herceptin); lapatinib (Tykerb) or AC-480.

Histone Deacetylase Inhibitors include, but are not limited to, vorinostat (Zolinza).

Exemplary hormones include, but are not limited to, tamoxifen (Soltamox; Nolvadex); raloxifene (Evista); megestrol (Megace); leuprolide (Lupron; Lupron Depot; Eligard; Viadur); fulvestrant (Faslodex); letrozole (Femara); triptorelin (Trelstar LA; Trelstar Depot); exemestane (Aromasin); goserelin (Zoladex); bicalutamide (Casodex); anastrozole (Arimidex); fluoxymesterone (Androxy; Halotestin); medroxyprogesterone (Provera; Depo-Provera); estramustine (Emcyt); flutamide (Eulexin); toremifene (Fareston); degarelix (Firmagon); nilutamide (Nilandron); abarelix (Plenaxis); or testolactone (Teslac).

Exemplary mitotic inhibitors include, but are not limited to, paclitaxel (Taxol; Onxol; Abraxane); docetaxel (Taxotere); vincristine (Oncovin; Vincasar PFS); vinblastine (Velban); etoposide (Toposar; Etopophos; VePesid); teniposide (Vumon); ixabepilone (Ixempra); nocodazole; epothilone; vinorelbine (Navelbine); camptothecin (CPT); irinotecan (Camptosar); topotecan (Hycamtin); amsacrine or lamellarin D (LAM-D).

Exemplary MTOR inhibitors include, but are not limited to, everolimus (Afinitor) or temsirolimus (Torisel); rapamune, ridaforolimus; or AP23573.

Exemplary multi-kinase inhibitors include, but are not limited to, sorafenib (Nexavar); sunitinib (Sutent); BIBW 2992; E7080; Zd6474; PKC-412; motesanib; or AP24534.

Exemplary serine/threonine kinase inhibitors include, but are not limited to, ruboxistaurin; eril/easudil hydrochloride; flavopiridol; seliciclib (CYC202; Roscovitrine); SNS-032 (BMS-387032); Pkc412; bryostatin; KAI-9803; SF1126; VX-680; Azd1152; Arry-142886 (AZD-6244); SCIO-469; GW681323; CC-401; CEP-1347 or PD 332991.

Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); CEP-701; SU5614; MLN518; XL999; VX-322; Azd0530; BMS-354825; SKI-606 CP-690; AG-490; WHI-P154; WHI-P131; AC-220; or AMG888.

Exemplary VEGF/VEGFR inhibitors include, but are not limited to, bevacizumab (Avastin); sorafenib (Nexavar); sunitinib (Sutent); ranibizumab; pegaptanib; or vandetinib.

Exemplary microtubule targeting drugs include, but are not limited to, paclitaxel, docetaxel, vincristin, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin and idarubicin.

Exemplary taxanes or taxane derivatives include, but are not limited to, paclitaxel and docetaxol.

Exemplary general chemotherapeutic, anti-neoplastic, anti-proliferative agents include, but are not limited to, altretamine (Hexalen); isotretinoin (Accutane; Amnesteem; Claravis; Sotret); tretinoin (Vesanoid); azacitidine (Vidaza); bortezomib (Velcade) asparaginase (Elspar); levamisole (Ergamisol); mitotane (Lysodren); procarbazine (Matulane); pegaspargase (Oncaspar); denileukin diftitox (Ontak); porfimer (Photofrin); aldesleukin (Proleukin); lenalidomide (Revlimid); bexarotene (Targretin); thalidomide (Thalomid); temsirolimus (Torisel); arsenic trioxide (Trisenox); verteporfin (Visudyne); mimosine (Leucenol); (1M tegafur—0.4 M 5-chloro-2,4-dihydroxypyrimidine-1 M potassium oxonate) or lovastatin.

Exemplary kinase inhibitors include, but are not limited to, Bevacizumab (targets VEGF), BIBW 2992 (targets EGFR and Erb2), Cetuximab/Erbitux (targets Erb1), Imatinib/Gleevic (targets Bcr-Abl), Trastuzumab (targets Erb2), Gefitinib/Iressa (targets EGFR), Ranibizumab (targets VEGF), Pegaptanib (targets VEGF), Erlotinib/Tarceva (targets Erb1), Nilotinib (targets Bcr-Abl), Lapatinib (targets Erb1 and Erb2/Her2), GW-572016/lapatinib ditosylate (targets HER2/Erb2), Panitumumab/Vectibix (targets EGFR), Vandetinib (targets RET/VEGFR), E7080 (multiple targets including RET and VEGFR), Herceptin (targets HER2/Erb2), PKI-166 (targets EGFR), Canertinib/CI-1033 (targets EGFR), Sunitinib/SU-11464/Sutent (targets EGFR and FLT3), Matuzumab/Emd7200 (targets EGFR), EKB-569 (targets EGFR), Zd6474 (targets EGFR and VEGFR), PKC-412 (targets VEGR and FLT3), Vatalanib/Ptk787/ZK222584 (targets VEGR), CEP-701 (targets FLT3), SU5614 (targets FLT3), MLN518 (targets FLT3), XL999 (targets FLT3), VX-322 (targets FLT3), Azd0530 (targets SRC), BMS-354825 (targets SRC), SKI-606 (targets SRC), CP-690 (targets JAK), AG-490 (targets JAK), WHI-P154 (targets JAK), WHI-P131 (targets JAK), sorafenib/Nexavar (targets RAF kinase, VEGFR-1, VEGFR-2, VEGFR-3, PDGFR-B, KIT, FLT-3, and RET), Dasatinib/Sprycel (BCR/ABL and Src), AC-220 (targets Flt3), AC-480 (targets all HER proteins, “panHER”), Motesanib diphosphate (targets VEGF1-3, PDGFR, and c-kit), Denosumab (targets RANKL, inhibits SRC), AMG888 (targets HER3), and AP24534 (multiple targets including Flt3).

Exemplary serine/threonine kinase inhibitors include, but are not limited to, Rapamune (targets mTOR/FRAP1), Deforolimus (targets mTOR), Certican/Everolimus (targets mTOR/FRAP1), AP23573 (targets mTOR/FRAP1), Eril/Fasudil hydrochloride (targets RHO), Flavopiridol (targets CDK), Seliciclib/CYC202/Roscovitrine (targets CDK), SNS-032/BMS-387032 (targets CDK), Ruboxistaurin (targets PKC), Pkc412 (targets PKC), Bryostatin (targets PKC), KAI-9803 (targets PKC), SF1126 (targets PI3K), VX-680 (targets Aurora kinase), Azd1152 (targets Aurora kinase), Arry-142886/AZD-6244 (targets MAP/MEK), SCIO-469 (targets MAP/MEK), GW681323 (targets MAP/MEK), CC-401 (targets JNK), CEP-1347 (targets JNK), and PD 332991 (targets CDK).

Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### Example 1: A Two-Tier Test Improves Risk of Recurrence Determination

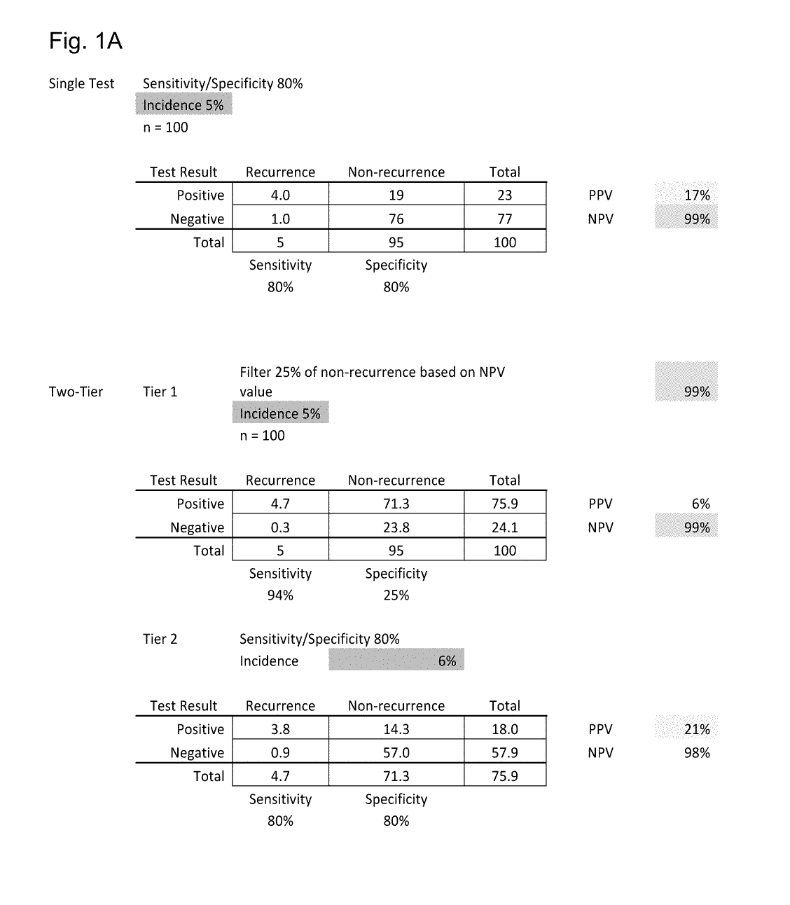

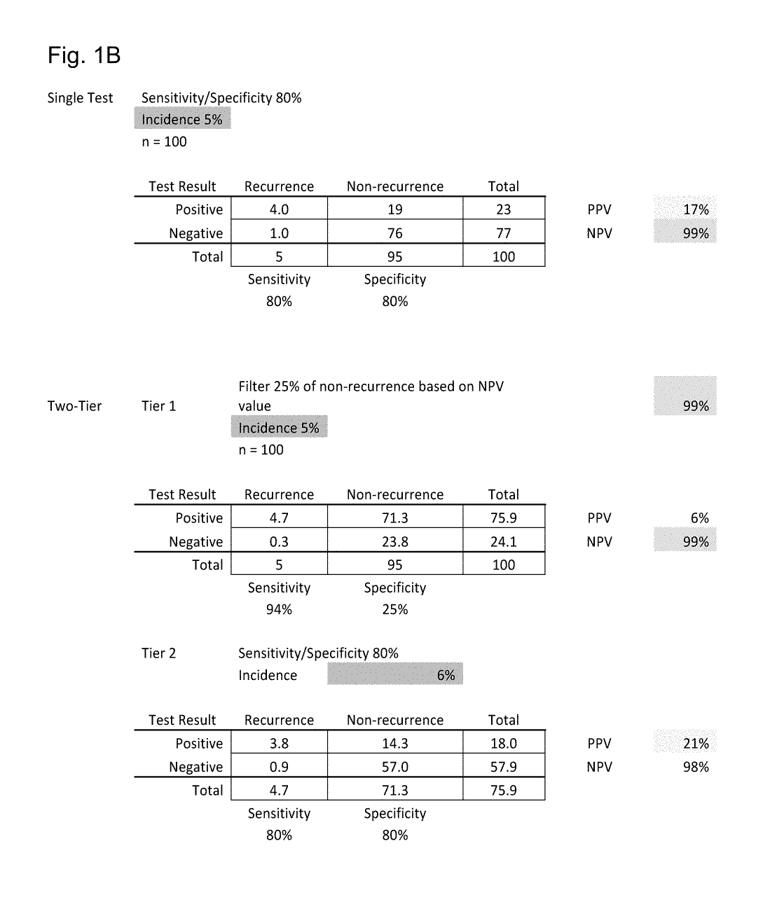

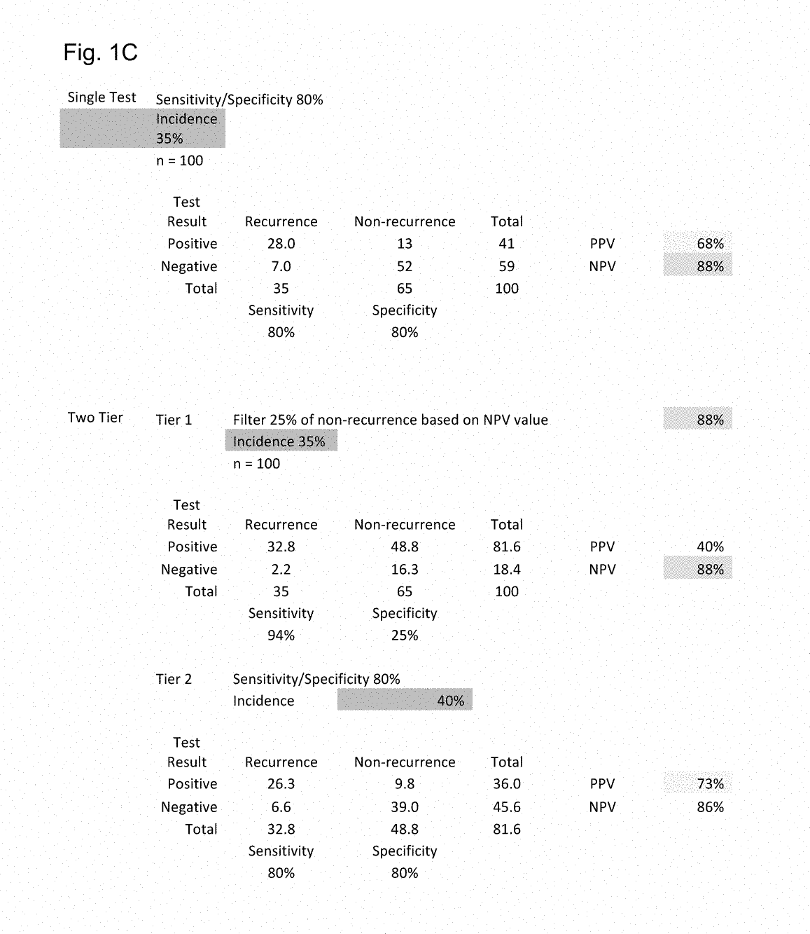

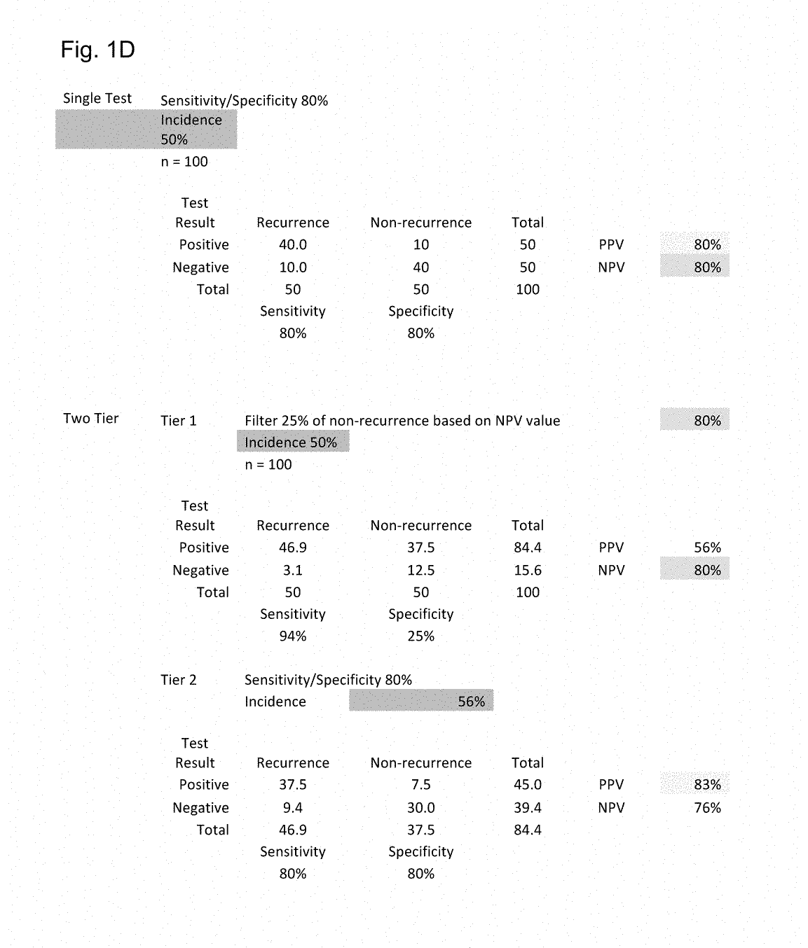

Models were developed to test the utility of a two-tier test over a single-tier test in classifying patients as test positive or test negative. Contingency tables generated from these models using hypothetical data are shown in FIGS. 1-3 and demonstrate the superior properties of a two-tier test over a single test.

First, a single test was performed using a population (n=100) with 5% incidence of recurrence and a hypothetical sensitivity and specificity of 80% (FIG. 1A). For this population, the NPV value was 99% and the PPV value was 17%. Next, a two-tier test was performed for the same population. In this test, the non-recurrent population the first tier test a hypothetical specify was set at 25% and all test negative cases were excluded (“filtered”) from the second test. In this example, the 25% filter removed 23.8 non-recurrent cases from tier two testing. Next, the false positive value was calculated and the false negative value was determined from the NPV value. In this example, the NPV value of the first tier of the test was 99%, the number of false negatives was 0.3, and the remaining test positive/true positive cases were calculated (4.7). For the second tier of the test, a contingency table was generated using the same 80% sensitivity/specificity values that were used in the single test. In the second tier of the test, the PPV value was 21%.

These tests were also performed on populations with 20%, 35%, or 50% incidence, respectively (FIGS. 1B, C, and D). In all cases, PPV values were higher in the two-tier test than in the single test. These data show that PPV values are increased in using a two-tier test and support the use of the two-tier test in patient populations with varied incidence (e.g. Stage I, II, or III cancer patients).

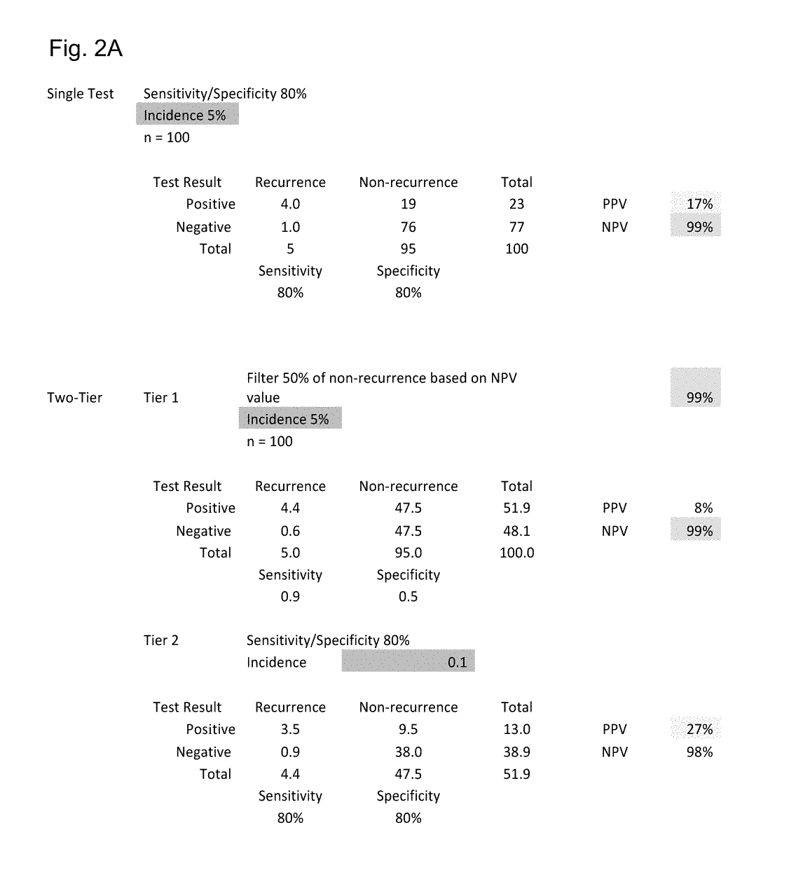

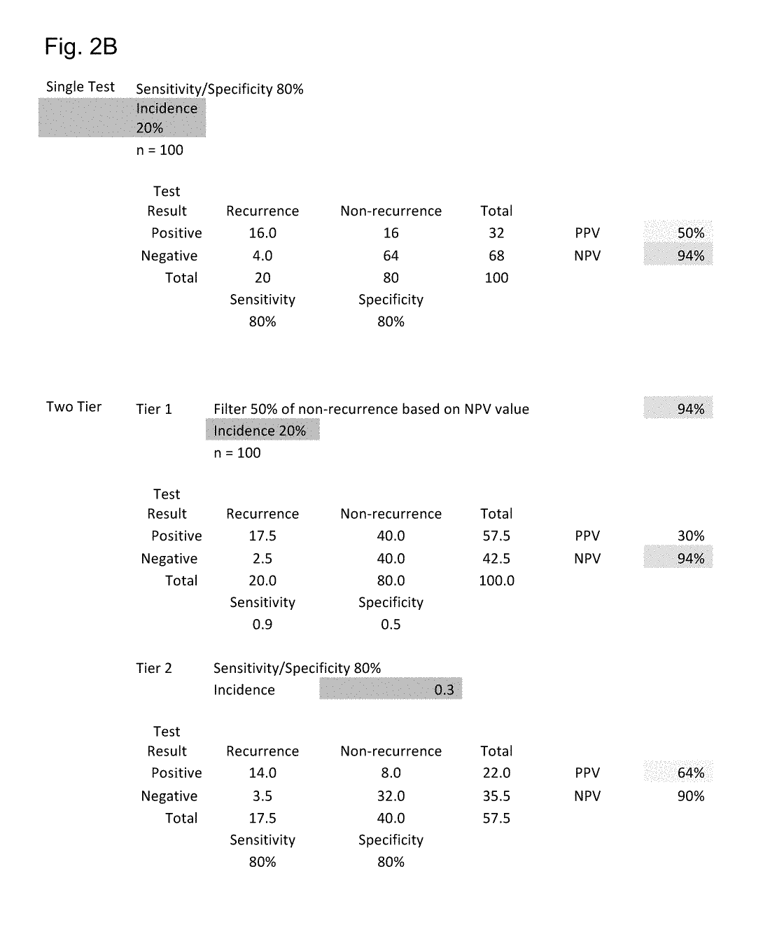

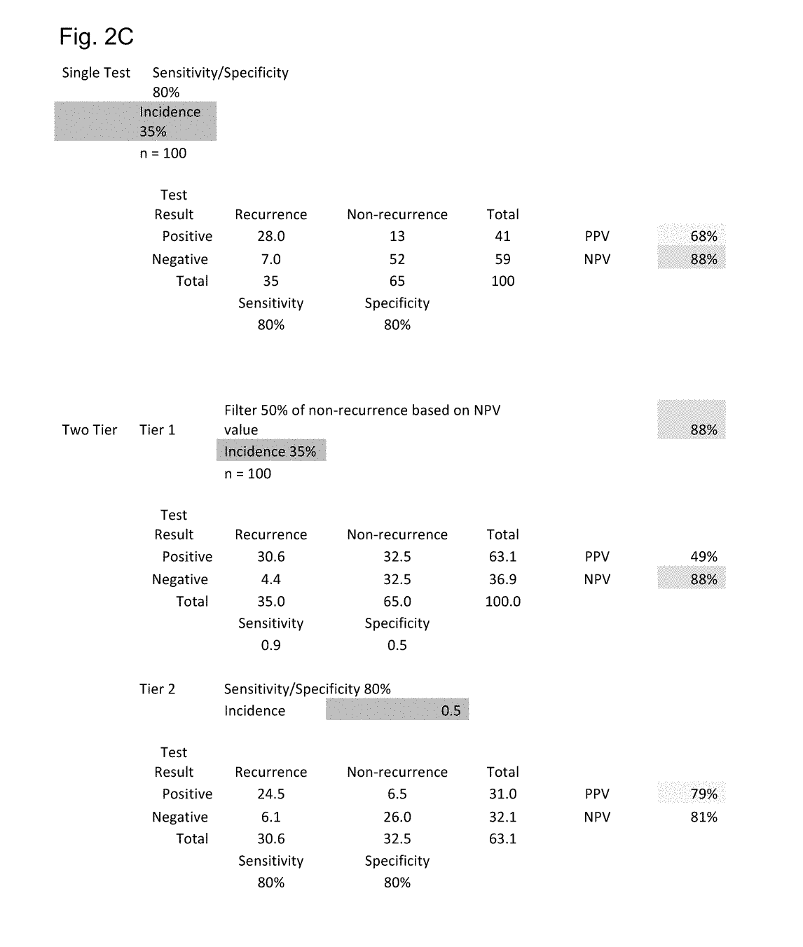

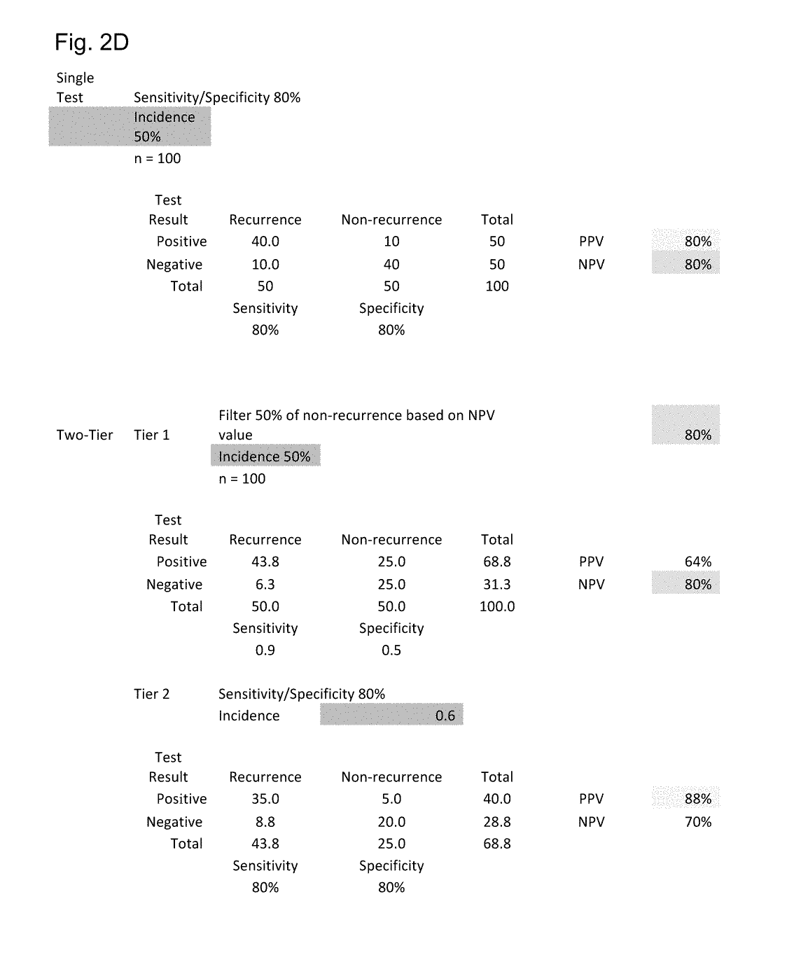

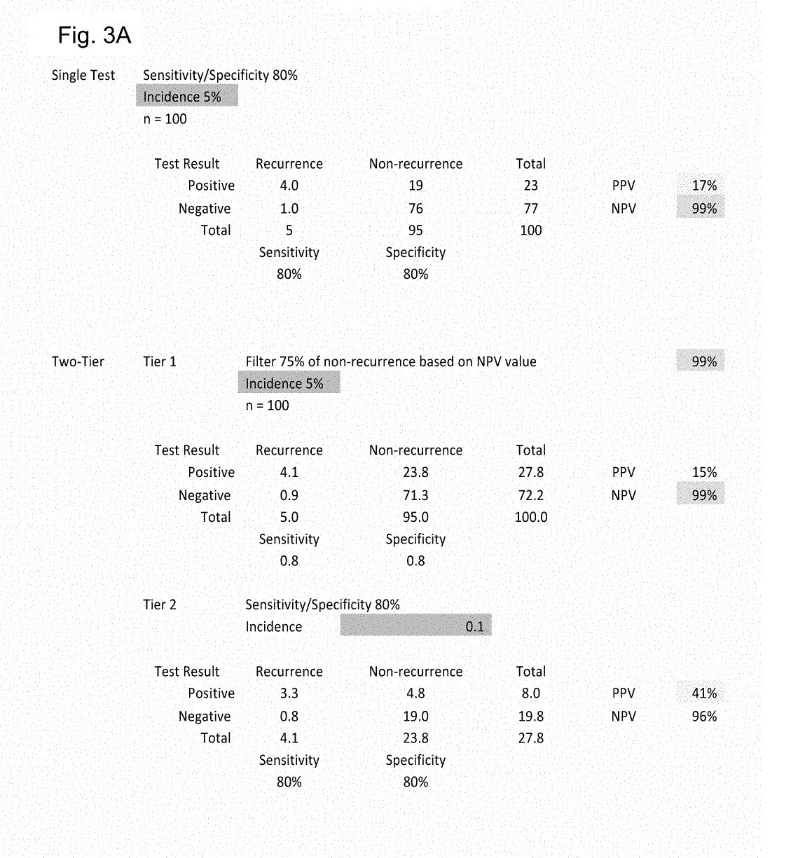

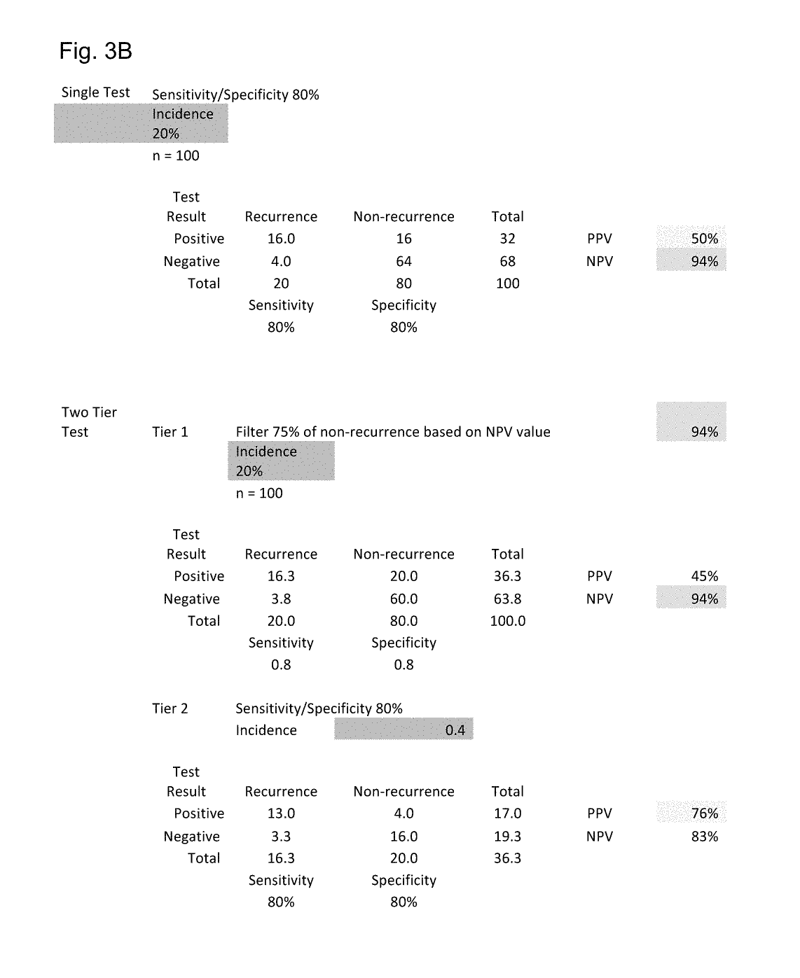

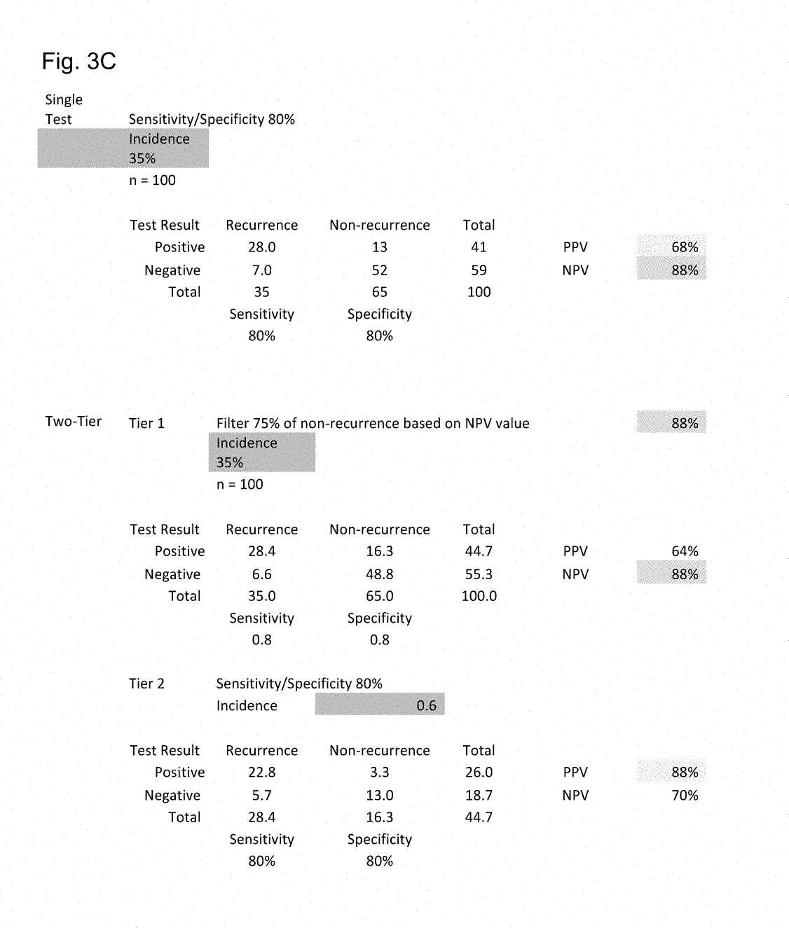

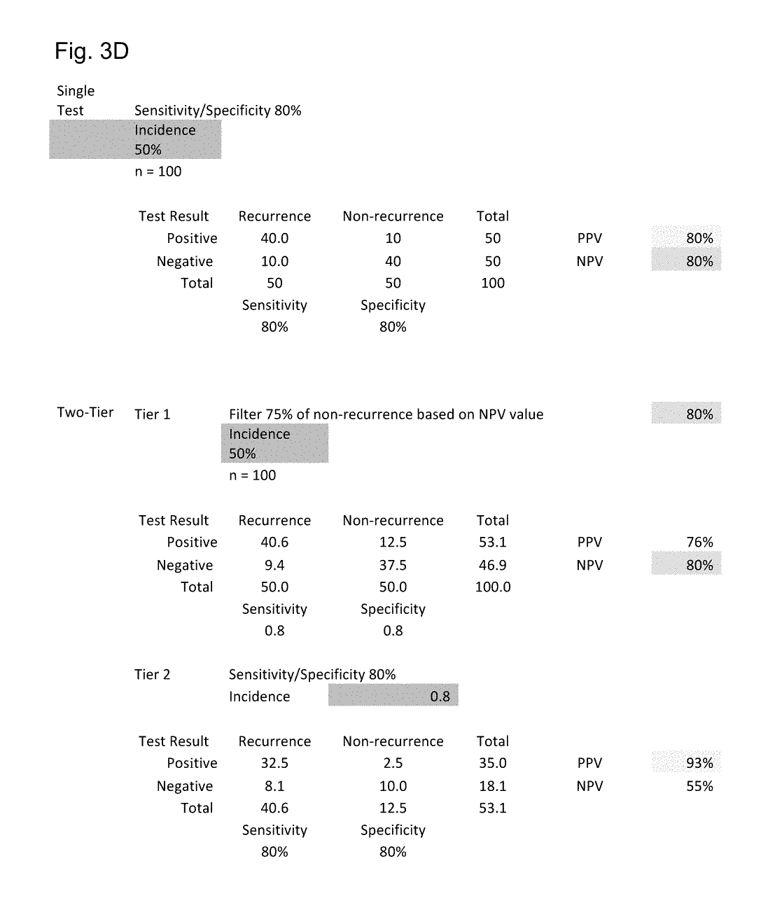

To further assess the utility of the two-tier test, additional models were generated by using specificity values of 50% or 75% for the tier one test, thus excluding 50% or 75% of the non-recurrent patients from the second tier respectively, and using the parameters described above (FIG. 2 and FIG. 3). In these models, PPV values were also higher in the two-tier test than in the single test, again demonstrating the utility of the two-tier test as compared to a single test and its utility in classifying patients across varied populations.

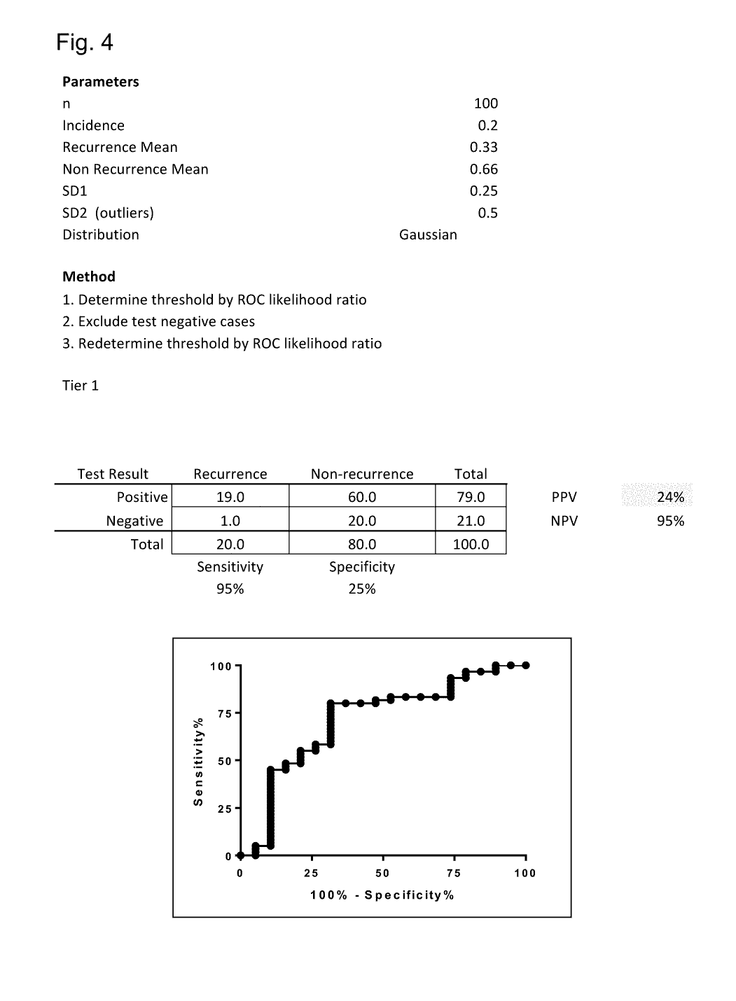

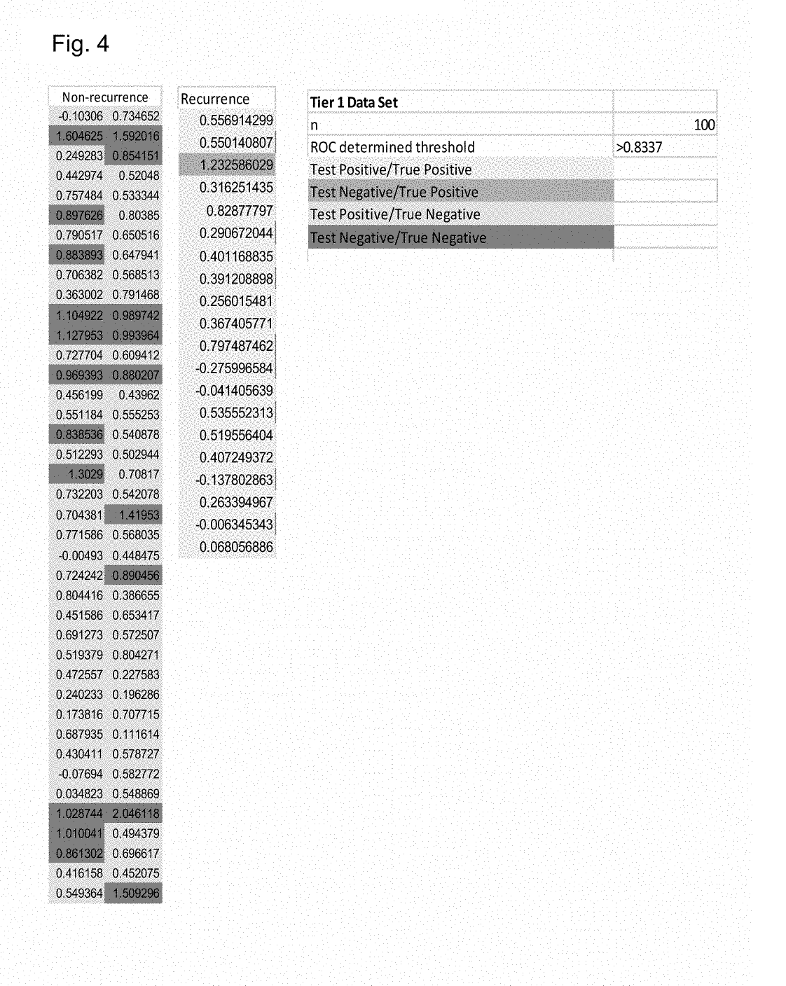

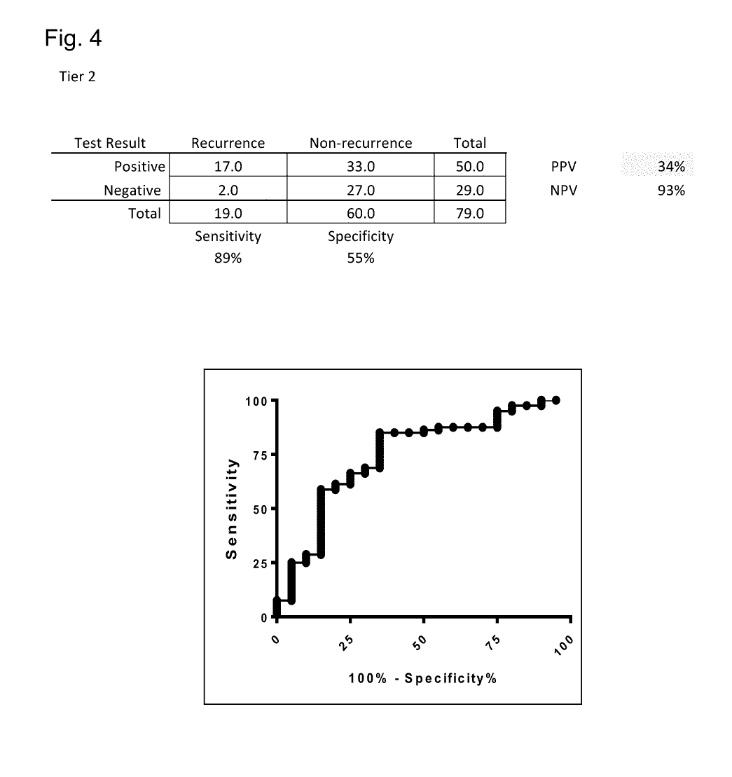

A second model was developed and assessed in a population (n=100) with 20% incidence of recurrence. Individual scores for each representative case in the population were randomly generated where the mean score of all recurrence cases was 0.33 and the mean score of all non-recurrence cases was 0.66, the standard deviation for all scores was 0.25 and distribution of scores was Gaussian. Next, receiver operating characteristic (ROC) curve analysis was used to determine an optimal threshold to discriminate recurrent and non-recurrent cases based on likelihood ratio. Next, a contingency table was generated from the threshold (FIG. 4). In this example, the ROC determined threshold was 0.8337; those patients above the threshold were considered to be “test negative” and excluded from the second tier of the test. Here, 21 test cases were excluded. Those patients subjected to the second tier of the test were considered to be “test positive” by the first tier test. For the second tier of the test, ROC analysis was repeated to generate a new threshold value. Here, the ROC determined threshold was 0.5652. Next, a contingency table for the second tier was calculated and generated a PPV value that was compared to the PPV value generated from the single test. Again, the two-tier test in this model generated a higher PPV value than that of the single test.

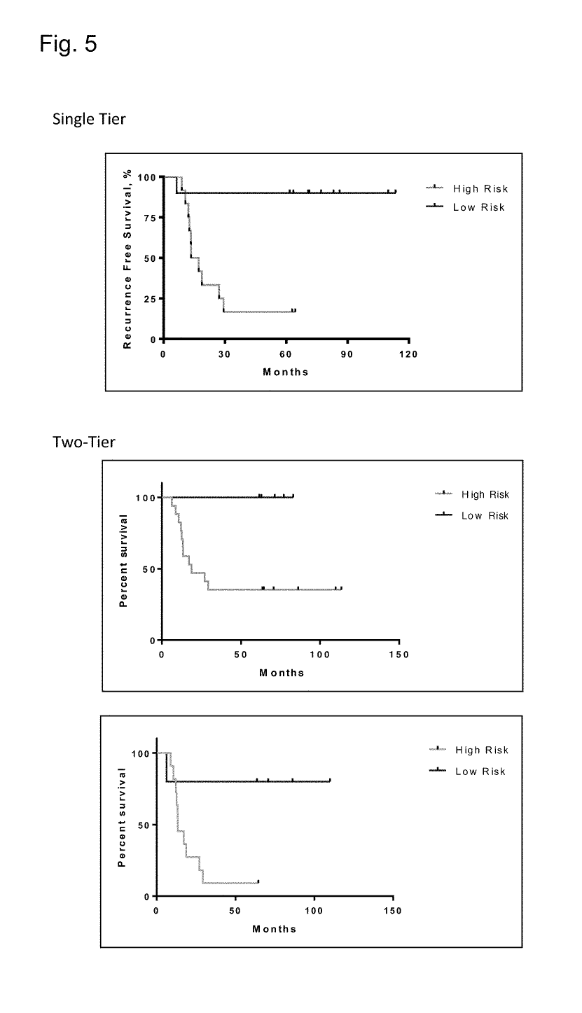

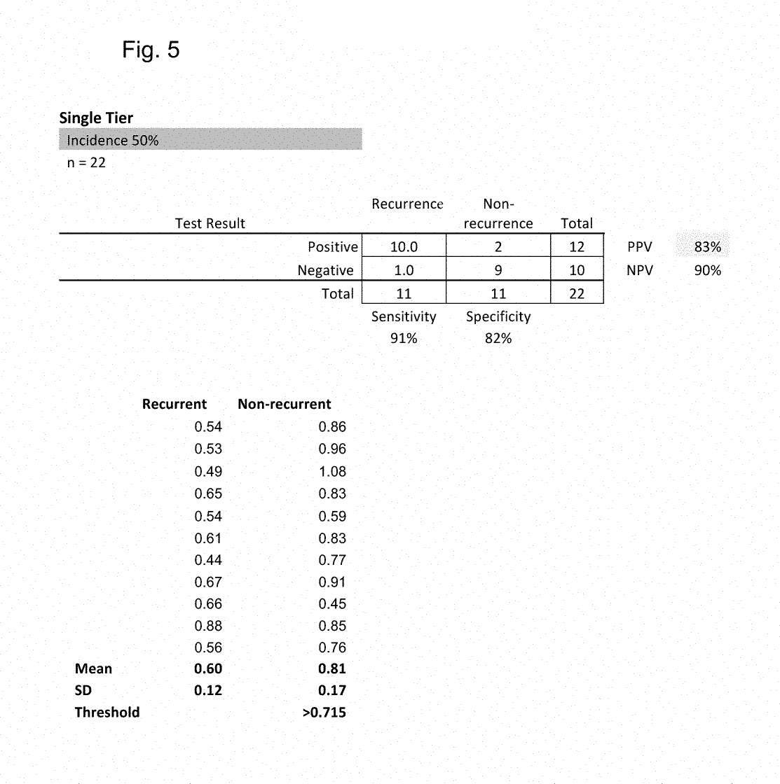

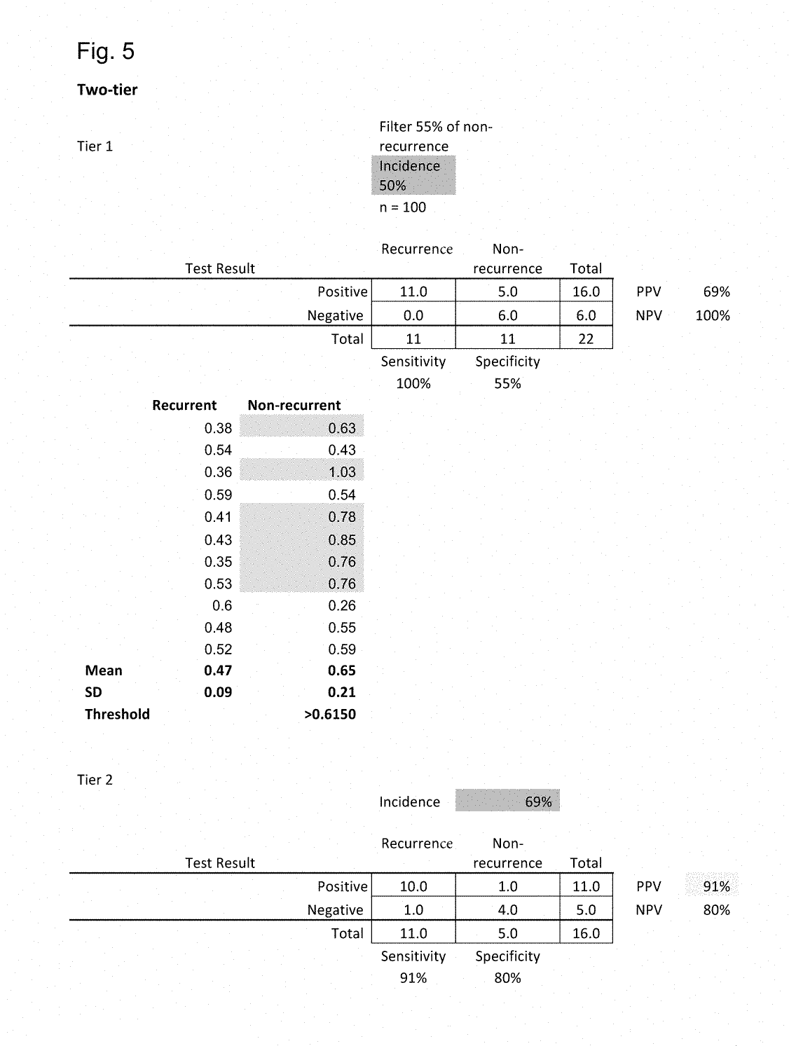

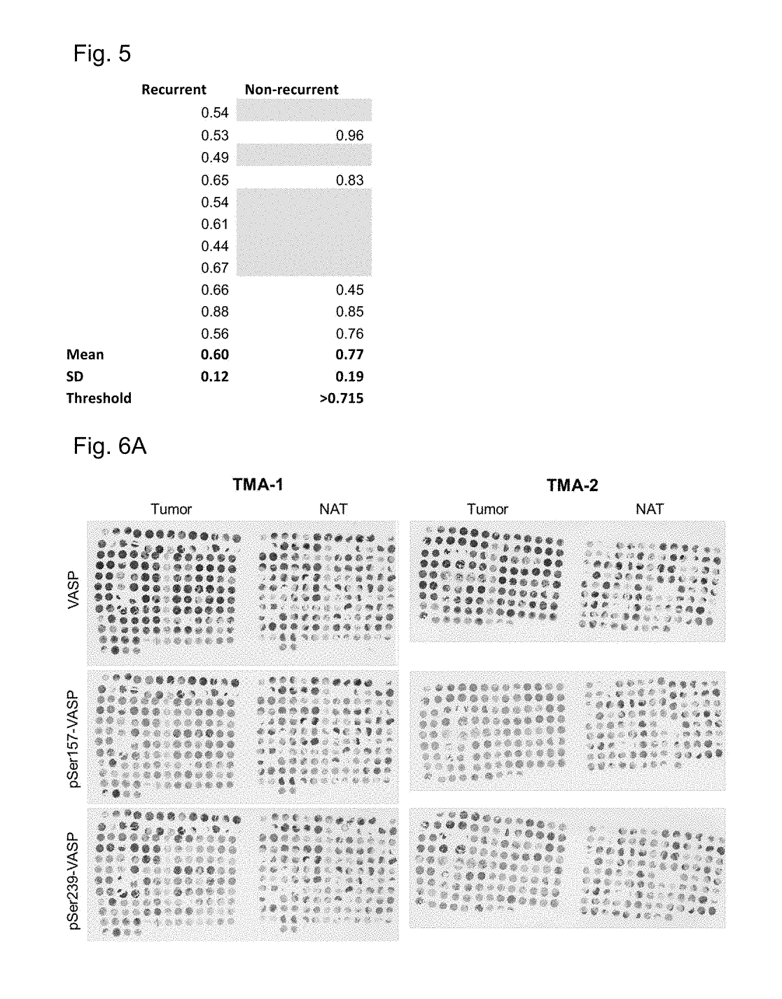

The utility of this model was further assessed with VASP biomarkers from patient data (FIG. 5). 22 tumors and matched NAT with T3 disease from a population with 50% incidence of recurrence was assessed. First, the relative tumor/NAT ratios of each VASP biomarker were calculated by applying the algorithm:

SQRT(CA239/NAT239×CA157/NAT157)/(CAvasp/NATvasp)̂2

This algorithm provided an index score for each patient investigated. ROC analysis of these scores identified a threshold value that optimally discriminated recurrent and non-recurrent patients (0.715). Then, a contingency table was generated from the threshold, and NPV and PPV values were calculated. For this single test, the PPV value was 83%. Next, a two-tier test was performed with the same population. For the first tier of the two-tier test, a different algorithm than the algorithm employed in the single test used to calculate index scores for each patient investigated were calculated by applying a different algorithm:

(CA157/CAvasp)/(NAT157NATvasp).

ROC analysis of these scores identified a threshold value that optimally discriminated recurrent and non-recurrent patients (0.6150). Here, the NPV value of the first tier was 100% and tier one test negative patients were excluded from tier two testing. For the second tier of the test, different index scores for each patient investigated were calculated by applying the algorithm employed in the single tier test described above. ROC analysis was repeated to generate a new threshold value. Here, the ROC determined threshold was 0.5652. Next, a contingency table for a PPV value (91%) was generated for this tier. Again, the two-tier test in this model generated a higher PPV value than that of the single test. These results demonstrate the clinical utility of the two-tier test in accurately classifying risk of recurrence in a clinical setting.

### Example 2: Materials and Methods

Patients.

For the tissue microarrays (TMAs), paraffin-embedded colorectal adenocarcinomas and respective normal adjacent tissues (NATs) from 119 patients homogeneously distributed along tumor-node-metastasis (TNM) pathological stages (Table 1) were obtained from the Department of Pathology, Anatomy and Cell Biology at Thomas Jefferson University (Philadelphia, Pa.), under a protocol approved by the Institutional Review Board (IRB).

TABLE 1
Clinicopathologic parameters of CRC patients in TMA study.

| Age (y) | | |
|---|---|---|
| Median (Range) | 66 | (28-91) |
| Gender (%) | | |
| Male | 56 | (47.1) |
| Female | 61 | (51.2) |
| ND | 2 | (1.7) |
| Race (%) | | |
| Caucasian | 67 | (56.3) |
| African American | 43 | (36.1) |
| Hispanic | 3 | (2.5) |
| ND | 6 | (5.1) |
| Tumor Site (%) | | |
| Right Colon | 46 | (38.6) |
| Transverse Colon | 9 | (7.6) |
| Left colon | 37 | (31.1) |
| Sigmoid | 17 | (14.3) |
| ND | 10 | (8.4) |
| TNM Stage^{a} (%) | | |
| 0 (Tis, N0M0) | 12 | (10.1) |
| I (T1-2, N0M0) | 24 | (20.2) |
| IIA (T3, N0M0) | 11 | (9.2) |
| IIB (T4, N0M0) | 20 | (16.8) |
| IIIA (T1-2, N1M0) | 12 | (10.1) |
| IIIB (T3-4, N1M0) | 12 | (10.1) |
| IIIC (T1-4, N2M0) | 13 | (10.9) |
| IV (T1-4, N0-2, M1) | 15 | (12.6) |
| Differentiation Grade (%) | | |
| Well | 9 | (7.6) |
| Moderate | 82 | (68.9) |
| Poor | 12 | (10.1) |
| ND | 16 | (13.4) |
| Lymph Node Metastasis (%) | | |
| Yes (N+) | 37 | (35.6) |
| No (N0) | 67 | (64.4) |
| Distant Metastasis (%) | | |
| Yes (M+) | 15 | (12.6) |
| No (M0) | 104 | (87.4) |

^{a}TNM (Tumor, Node, Metastasis) annotations indicate: Tis, limited to mucosa (carcinoma in situ); T1, limited to submucosa; T2, invading the muscularis propria; T3, invading the serosa; T4, invading adjacent organs; N0, no lymph nodes involvement; N1, metastasis in 1-3 lymph nodes; N2, metastasis in ≥4 lymph nodes; M0, no distant metastasis; M1, metastasis at distant organs.
ND, not determined.

Tissue blocks, sorted by TNM stage, were processed and correspondent tissue cores of 0.7 mm in diameter were collected from regions of interest and assembled in duplicate into 2 TMA blocks, TMA-1 and TMA-2, containing a total of 150 and 118 cores respectively (see below for detail). For whole-tissue section studies, clinical residual tissues from 22 stage II, T3 patients with matched colon adenocarcinomas and NAT (mounted as whole-tissue sections) and clinical outcomes data (≥5 yr follow-up) were obtained from the Mayo Clinic under an IRB-annroved protocol (Table 21.

TABLE 2
Clinicopathologic parameters of CRC patients
in whole tissue section study.

| Age (y) | | |
|---|---|---|
| Median (Range) | 62 | (48-81) |
| Gender (%) | | |
| Male | 12 | (54.5) |
| Female | 10 | (45.5) |
| Race (%) | | |
| Caucasian | 20 | (90.9) |
| ND | 2 | (9.1) |
| Tumor Site (%) | | |
| Right Colon | 5 | (22.7) |
| Transverse Colon | 5 | (22.7) |
| Left colon | 3 | (13.7) |
| Sigmoid | 9 | (40.9) |
| TNM Stage* (%) | | |
| IIA (T3, N0M0) | 22 | (100) |
| Differentiation Grade (%) | | |
| Well | 4 | (18.2) |
| Moderate | 17 | (77.3) |
| Poor | 1 | (4.5) |
| Lymph Nodes Examined | | |
| Median (Range) | 21.5 | (8-44) |
| Chemotherapy (%) | | |
| Yes | 0 | (0) |
| No | 22 | (100) |

*TNM (Tumor, Node, Metastasis) annotations indicate: T3, invading the serosa; N0, no lymph node involvement: M0, no distant metastasis.
ND, not determined

Tissue Microarrays.

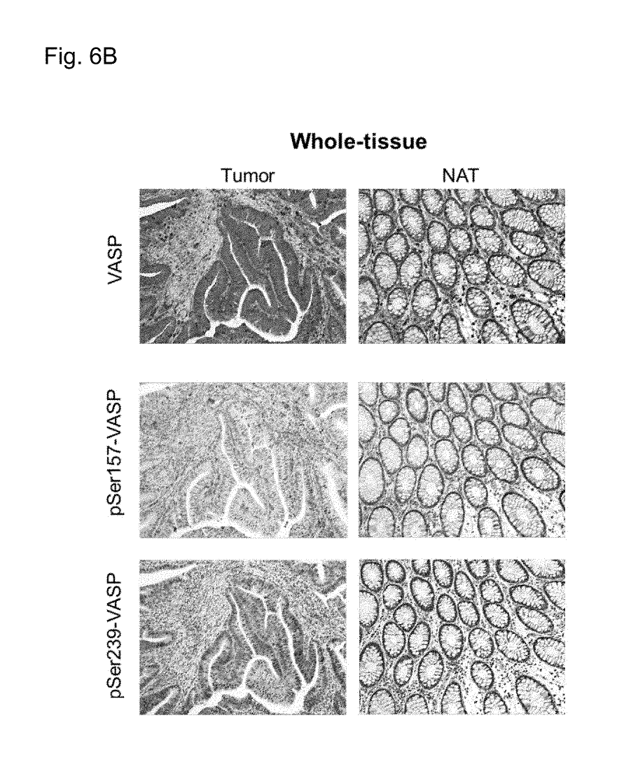

Each TMA block contained 2 tissue sectors, the Tumor grid and the correspondent NAT grid (FIG. 6). TMA-1 was constructed with 67 low TNM stage cases (from top-to-bottom: 12 stage 0, 24 stage I and 31 stage II), while TMA-2 contained 52 high TNM stages (from top-to-bottom: 37 stage III and 15 stage IV). Normal colorectal tissue controls from non-cancer patients were also allocated (in duplicate) in the first 6 positions (from top left corner) of each tissue sector, and served as the internal positive controls. Moreover, ten (in TMA-1) and eight (in TMA-2) tissue cores from human placenta from de-identified donors were allocated in vertical positions in the middle (number 7) column, starting from the second row, of each sector and served as the negative control samples. Then, 4 μm tissue sections were cut from each TMA, mounted on microscope slides and subjected to immunohistochemistry (IHC). Following standard pathological processing, insufficient or poorly processed tissue cores resulted in 2 (1.7%) patients lacking any relevant tissue, and 20 (16.8%) cases with 1-3 missing core pairs (in Tumor and/or NAT). Incomplete cases were also included in the analyses, which consequently translated in different total numbers of each biomarker evaluated (as indicated in brief description of drawings).

Immunohistochemistry.

IHC staining was performed with antibodies to human VASP (SC-46668, Santa Cruz, Santa Cruz, Calif.), pSer157-VASP (SC101818, Santa Cruz) or pSer239-VASP (SAB4300129, Sigma Aldrich, St. Louis, Mo.). Following sequential steps of deparaffinization, rehydratation and antigen retrieval, TMA slides were subjected to serial incubations with primary antibodies (VASP, 1:1000; pSer157-VASP, 1:100; pSer239-VASP, 1:500), appropriate secondary antibodies and the DAB reporter system (Vector Laboratory, Burlingame, Calif.). Then, the membranous and cytoplasmic staining intensity of each VASP marker (evaluated in epithelial cell compartments only) was semiquantitatively scored by two blinded clinical pathologists on a 0-to-3 scale (0, absent; 1, weak; 2, moderate; 3, strong). In the whole-tissue section study, slides were processed using the Bond Polymer Refine Detection (DS9800, Leica) staining kit on a Bond automated stainer (Leica). IHC was performed according to the following conditions: VASP, 1:3000 (with BOND Epitope Retrieval Solution 2); pSer157-VASP, 1:200 (with BOND Epitope Retrieval Solution 2), and pSer239-VASP, 1:500 (with BOND Epitope Retrieval Solution 1). Levels of VASP biomarkers in epithelial membranous and cytoplasmic compartments were calculated semiquantitatively by two blinded clinical pathologists as H-scores, that measure staining intensity (0, absent; 1, weak; 2, moderate; 3, strong) in combination with the percentage of cells staining positively [H-score=(3×% cells)+(2×% cells)+(1×% cells)].

Statistical Analysis.

Comparisons of VASP biomarker expression in Tumor vs. NAT, invasive vs. preinvasive lesions or N+ vs. N0 disease were evaluated by two-sided Student's t-tests. Pathologists' H-scoring comparisons were evaluated with the Spearman Correlation test. Receiver Operating Characteristic analysis was employed to determine optimal thresholds that discriminated low-risk and high-risk patients, and time to recurrence was analyzed using the Kaplan-Meier estimator of the survival curves. Test-positive patients had documented disease recurrence and test-negative patients were defined as recurrence-free for ≥5 years following initial surgery, and were censored on the date of last follow-up. The difference in time to recurrence between test-negative and test-positive patients was evaluated using the two-sided log-rank test. Cox proportional hazard models were used to determine hazard ratios (HRs) and 95% coefficient intervals (Cis). The algorithm, I(pSer157-VASP Tumor/NAT×pSer239-VASP Tumor/NAT)/(VASP Tumor/NAT)²′, provided a single index score to evaluate combined biomarker ratios. All statistical analyses were performed with GraphPad Prism software (Version 7).

### Example 3: Differential Expression of VASP and its Phosphorylated Forms in Primary Human CRC Tumors

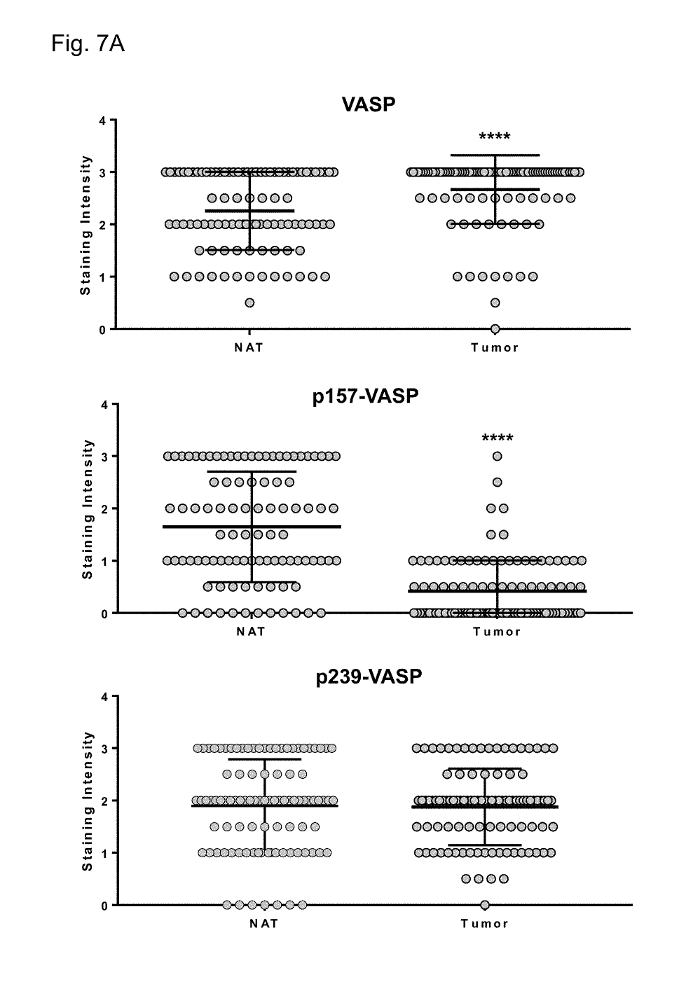

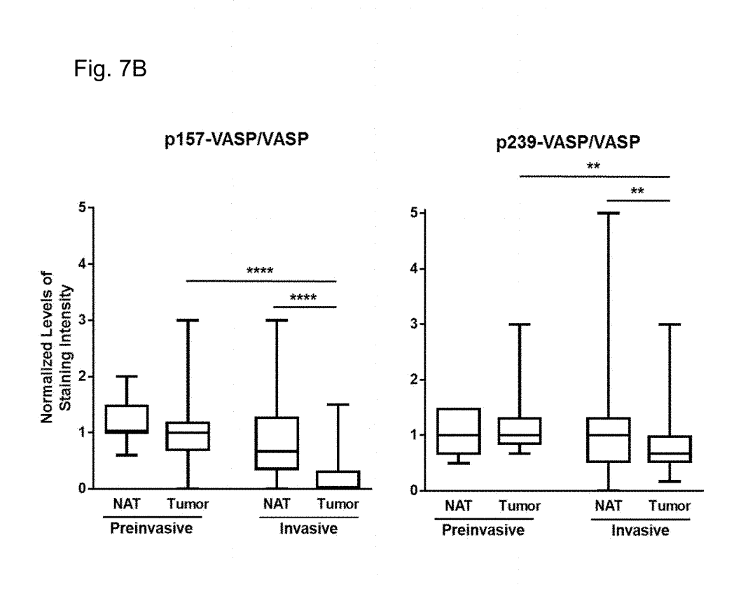

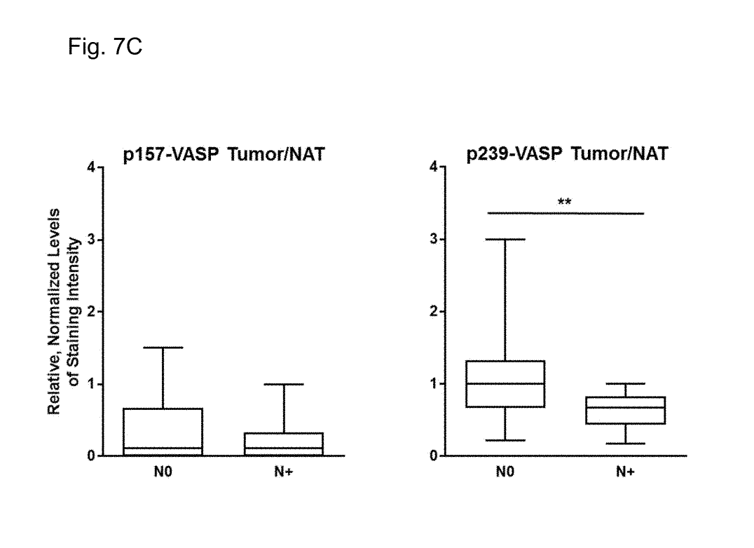

The relationship between VASP, pSer¹⁵⁷-VASP and pSer²³⁹-VASP in primary human CRC tumors with disease progression was evaluated. Tissue microarrays (TMAs) containing 119 primary CRC tumors and matched normal adjacent tissue (NAT) specimens were subjected to immunohistochemistry (IHC) for each VASP biomarker (VASP, pSer¹⁵⁷-VASP or pSer²³⁹-VASP), and semi-quantitative scoring was performed by pathologists blinded to clinical data (0-3 scale; Table 1 and FIG. 6A). Compared to matched NATs, tumors exhibited significant expression changes in VASP (upregulation) and pSer¹⁵⁷-VASP (downregulation; FIG. 7A). However, following normalization, where staining intensity scores for pSer¹⁵⁷-VASP and pSer²³⁹-VASP were divided by the score for VASP, both phosphorylated VASP forms were significantly downregulated in adenocarcinomas compared to matched NAT, or compared to non-matched carcinoma in-situ (FIG. 7B). Moreover, analysis of normalized relative levels (Tumor/NAT) of VASP phosphorylated forms revealed that pSer²³⁹-VASP was also significantly decreased in lymph node positive (N+) compared to node negative (N0) CRCs (FIG. 7C). These data suggest that differential expression of VASP and its phosphorylated forms are associated with CRC invasion and metastasis to regional lymph nodes.

### Example 4: Analysis of VASP Biomarkers Using a Two-Tier Test Improves Risk of Disease Progression and Risk of Recurrence Determination in CRC Patients

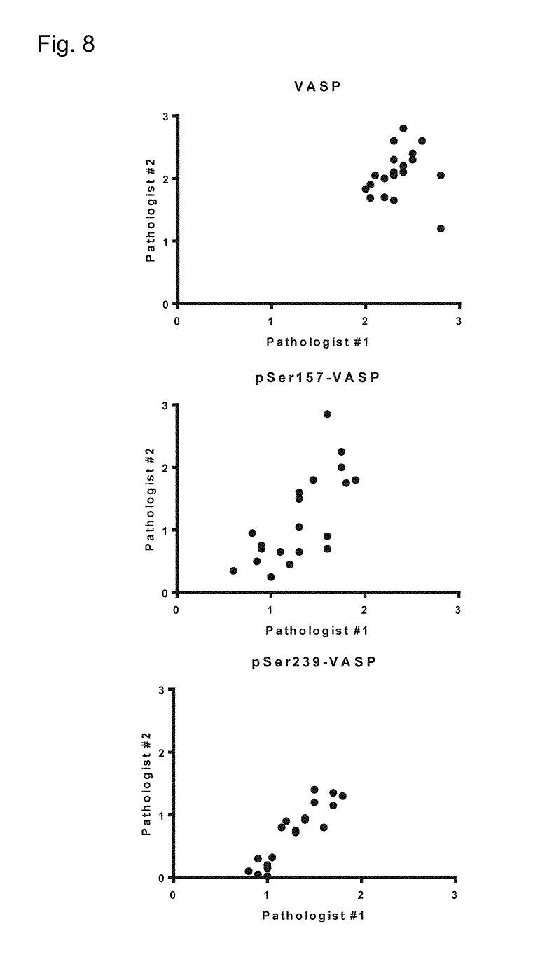

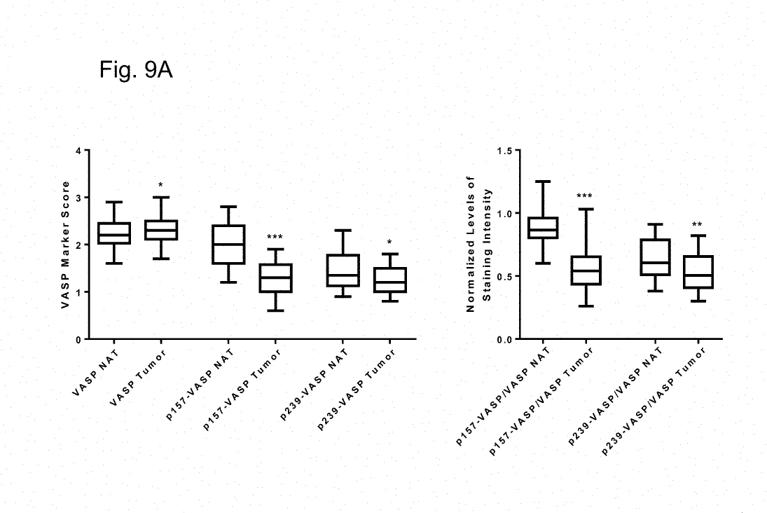

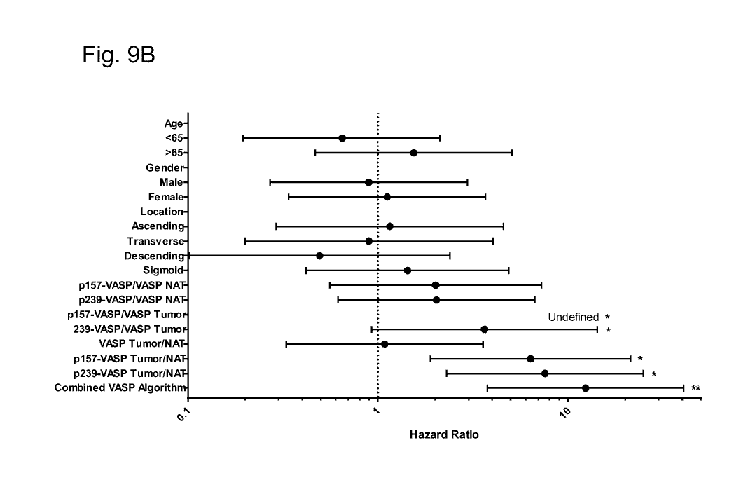

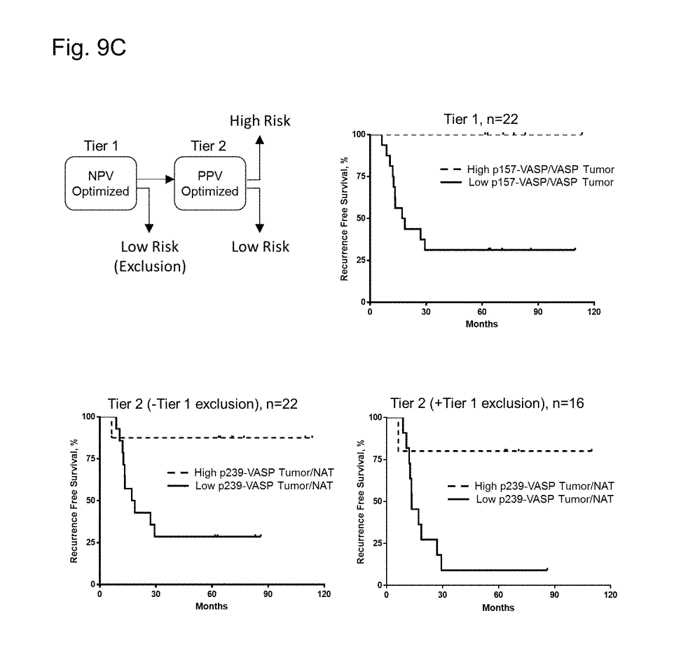

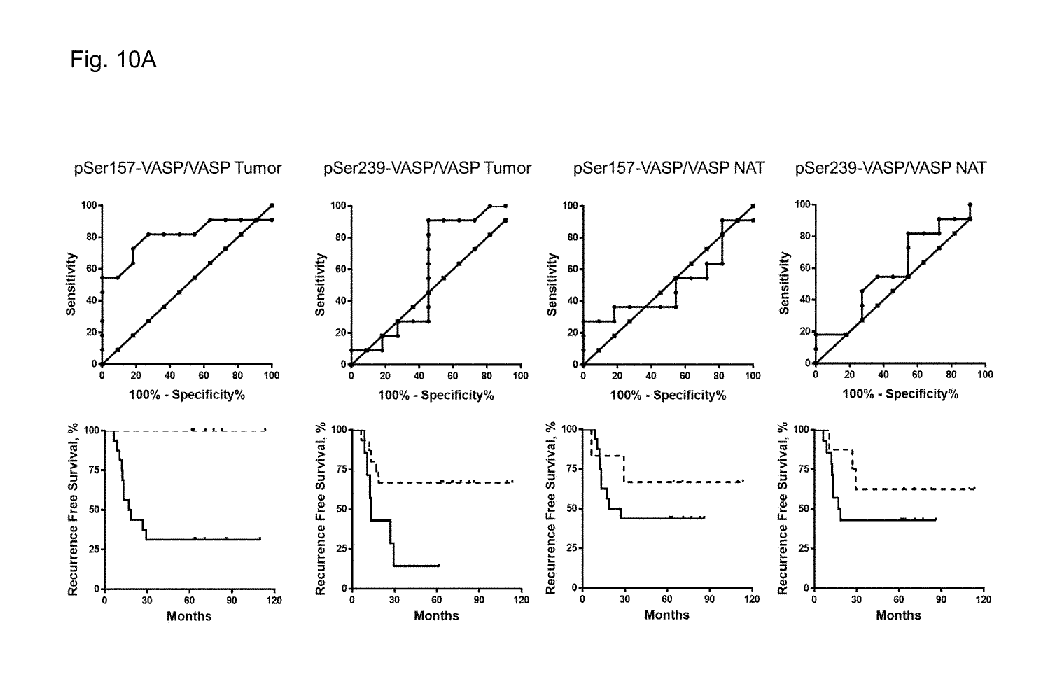

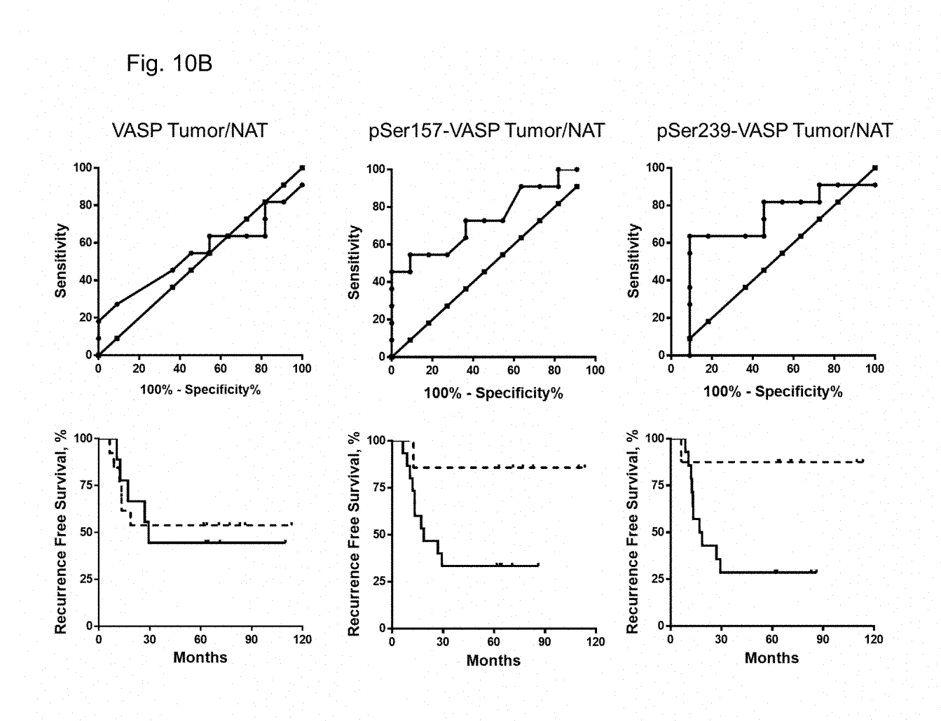

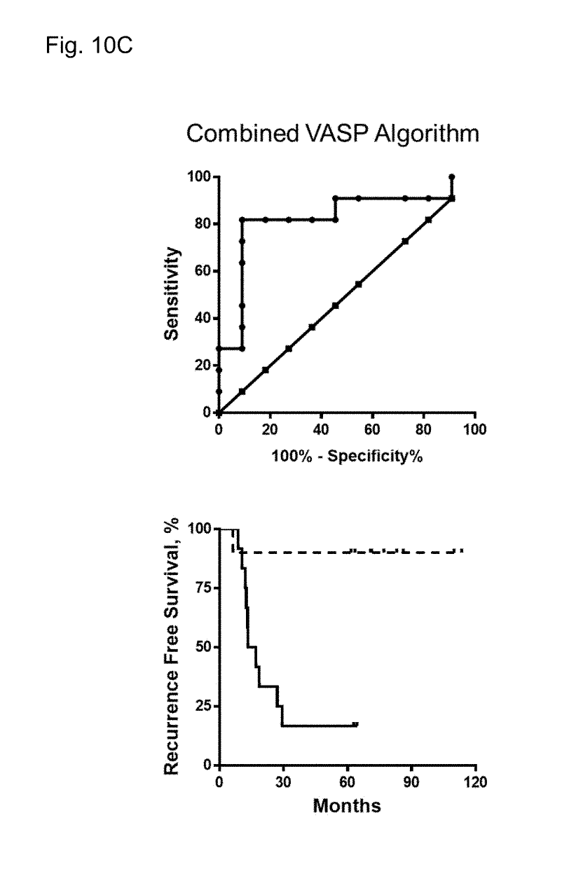

To investigate potential clinical utility of VASP biomarkers, a pilot study was performed employing tissues from 22 stage II (T3N0) CRC patients comprising primary tumors and matched NATs (mounted as whole-tissue sections). Expression of VASP biomarkers was analyzed in relationship to clinical outcome data (≥5 yr follow-up). The patient cohort was selected as chemotherapy-naive, well-balanced for tumor site and grade distribution, and enriched for tumor recurrence (55%; Table 2). Following IHC staining for VASP, pSer157-VASP or pSer239-VASP, a semi-quantitative H-scoring system was employed (FIG. 6B). Independent scores from two blinded pathologists were highly correlated, suggesting that staining intensity quantification of VASP biomarkers is a reliable and objective measurement (FIG. 8). Importantly and compared to NATs, tumors exhibited significant upregulation of VASP and downregulation of absolute or VASP-normalized levels of both pSer157-VASP and pSer239-VASP (FIG. 9A), confirming the observations from TMA studies. Then, VASP-normalized values of pSer157-VASP and pSer239-VASP, and relative tumor/NAT ratios of each VASP biomarker were calculated and Receiver Operating Characteristic (ROC) analysis performed to identify threshold values that optimally discriminate between recurrence and recurrence-free survival (FIG. 10). Further, hazard ratios (HRs) and 95% confidence intervals (CIs) were calculated using Cox proportional hazards models and Kaplan-Meier survival curves were generated (FIG. 9B, FIG. 10). VASP-normalized pSer157-VASP and pSer239-VASP values in tumors, but not NAT, significantly discriminated by recurrence and recurrence-free survival among the cohort, with HRs undefined for pSer157-VASP (due to absence of recurrence in the low-risk group), and of 3.6 (95% CI, 0.9-14.3; p=0.02) for pSer239-VASP (FIG. 10A, FIG. 9B). Relative tumor/NAT values of pSer157-VASP and pSer239-VASP, but not VASP, also significantly discriminated patient risk groups (FIG. 10B) and exhibited HRs of 6.3 (95% CI, 1.9-21.4; p=0.04) and 7.6 (95% CI, 2.3-25; p=0.02), respectively (FIG. 9B). Unlike VASP biomarkers, no significant associations were observed between traditional clinicopathological parameters, including age, sex or primary tumor site (FIG. 9B). Importantly, integration of multiple VASP biomarker ratios into algorithms provided single index scores that were found to more accurately discriminate between recurrence and recurrence-free survival than did individual ratios. ROC analysis of one such algorithm (FIG. 10C) resulted in an optimally-discriminating threshold (AUC=0.83; 95% C.I., 0.63 to 1.02; p=0.009) with an HR of 12.4 (95% CI, 3.8-40.6; p=0.002; FIG. 9B).

Finally, VASP biomarker ratios were applied in sequence, employing a novel, two-tiered model developed to optimize negative and positive predictive values (NPV, PPV) (FIG. 9C). Here, a Tier-1 stratification is applied based on optimization of NPV. Low risk patients identified by Tier-1 are then excluded from Tier-2 stratification. In this way, true negative depletion and the subsequent increased incidence of true positives in the Tier-2 cohort introduce a bias for higher PPV performance. VASP-normalized pSer157-VASP tumor ratio was selected as the Tier-1 test based on the high NPV exhibited (100%), while the pSer239-VASP tumor/NAT ratio (PPV, 72%) was employed in Tier-2. The PPV performance of the Tier-2 VASP test greatly improved (to 91%) following patient (n, 6) exclusion in Tier-1 (FIG. 9C). Altogether, these observations suggest that VASP biomarkers are associated with disease progression and recurrence risk in CRC patients, and may be configured to optimize clinically relevant measures such as NPV and PPV.

We claim:

1. A method for providing a clinical assessment of a subject in need thereof comprising
a) measuring the amount of at least one first biomarker in at least one first sample from the subject to generate a first score;
b) comparing the first score to a first predetermined threshold to determine if the subject is test positive or test negative, wherein the predetermined threshold has a negative predictive value of at least 80%;
c) providing a clinical assessment, wherein if the subject is determined to be test negative, the clinical assessment comprises recommending the subject is excluded from treatment;
d) measuring the amount of at least one second biomarker in at least one second sample from a subject determined to be test positive in step (b) to generate a second score;
e) comparing the second score to a second predetermined threshold to determine if the subject is test positive for the second predetermined threshold, wherein the predetermined threshold has a positive predictive value of at least 40%; and
f) providing a clinical assessment, wherein if the subject is determined to be test positive in step (e), the clinical assessment comprises recommending the subject receive treatment.

2. The method of claim 1, wherein the at least one first biomarker and the at least one second biomarker are the same.

3. The method of claim 1, wherein the at least one first biomarker and the at least one second biomarker are different.

4. The method of claim 1, wherein the at least one first biomarker or the at least one second biomarker is an amino acid molecule, a nucleic acid molecule, or a combination thereof.

5. The method of claim 1, wherein the at least one first sample and the at least one second sample are the same.

6. The method of claim 1, wherein the at least one first sample and the at least one second sample are different.

7. The method of claim 1, wherein the at least first sample or the at least second sample is a tumor tissue, normal tissue, normal tissue adjacent to a tumor, saliva, plasma, blood, serum, spinal fluid, lymphatic fluid, urine or a combination thereof.

8. The method of claim 7, wherein the at least first sample or the at least second sample is tumor tissue.

9. The method of claim 8, wherein the tumor tissue is epithelial tumor tissue.

10. The method of claim 1, wherein the first score and the second score is the same.

11. The method of claim 1, wherein the first score and the second score is different.

12. The method of claim 1, wherein the first score, the second score, or both the first score and the second score, are calculated using an algorithm.

13. The method of claim 1, wherein the first predetermined threshold and the second predetermined threshold is the same.

14. The method of claim 1, wherein the first predetermined threshold and second predetermined threshold is different.

15. The method of claim 1, wherein the first predetermined threshold, the second predetermined threshold, or both the first predetermined threshold and the second predetermined threshold, are calculated using an algorithm.

16. The method of claim 1, wherein the first predetermined threshold, the second predetermined threshold, or both the first predetermined threshold and the second predetermined threshold, have a sensitivity of at least 80%.

17. The method of claim 1, wherein the first predetermined threshold, the second predetermined threshold, or both the first predetermined threshold and the second predetermined threshold, have a sensitivity of at least 90%.

18. The method of claim 1, wherein the first predetermined threshold, the second predetermined threshold, or both the first predetermined threshold and the second predetermined threshold, have a sensitivity of at least 95%.

19. The method of claim 1, wherein the first predetermined threshold, the second predetermined threshold, or both the first predetermined threshold and the second predetermined threshold, have a specificity of at least 40%.

20. The method of claim 1, wherein the first predetermined threshold, the second predetermined threshold, or both the first predetermined threshold and the second predetermined threshold, have a specificity of at least 50%.

21. The method of claim 1, wherein the first predetermined threshold, the second predetermined threshold, or both the first predetermined threshold and the second predetermined threshold, have a specificity of at least 75%.

22. The method of claim 1, wherein the first predetermined threshold, the second predetermined threshold, or both the first predetermined threshold and the second predetermined threshold, have a specificity of at least 80%.

23. The method of claim 1, wherein the first predetermined threshold, the second predetermined threshold, or both the first predetermined threshold and the second predetermined threshold, have a specificity of at least 90%.

24. The method of claim 1, wherein the first predetermined threshold, the second predetermined threshold, or both the first predetermined threshold and the second predetermined threshold, have a specificity of at least 95%.

25. The method of claim 1, wherein the first predetermined threshold has a negative predictive value of at least 85%.

26. The method of claim 1, wherein the first predetermined threshold has a negative predictive value of at least 90%.

27. The method of claim 1, wherein the first predetermined threshold has a negative predictive value of at least 95%.

28. The method of claim 1, wherein the second predetermined threshold has a positive predictive value of at least 50%.

29. The method of claim 1, wherein the second predetermined threshold has a positive predictive value of at least 75%.

30. The method of claim 1, wherein the second predetermined threshold has a positive predictive value of at least 80%.

31. The method of claim 1, wherein the second predetermined threshold has a positive predictive value of at least 90%.

32. The method of claim 1, wherein the second predetermined threshold has a positive predictive value of at least 95%.

33. The method of claim 1, wherein the subject was previously treated for cancer.

34. The method of claim 1, wherein the subject presents with disease symptoms.

35. The method of claim 1, wherein the subject asymptomatic.

36. The method of claim 1, wherein the clinical assessment is risk of recurrence of cancer.

37. The method of claim 36, wherein the test negative subject excluded from treatment has a low risk of recurrence of cancer.

38. The method of claim 36, wherein the test positive subject recommended to receive treatment has a high risk of recurrence of cancer.

39. The method of claim 1, wherein the treatment comprises chemotherapy, immunotherapy, radiotherapy, or a combination thereof.

40. The method of claim 1, further comprising administering a treatment comprising chemotherapy, immunotherapy, radiotherapy, or a combination thereof, to a subject recommended to receive treatment.

41. A method for providing a clinical assessment of a subject in need thereof comprising
a) measuring the amount of at least one first biomarker in at least one first sample and the amount of at least one second biomarker in at least one second sample from the subject to generate a first score, wherein one of the at least first sample and at least second sample is tumor tissue and at least one of the at least first sample and at least second sample is normal tissue adjacent to the tumor tissue;
b) comparing the first score to a first predetermined threshold to determine if the subject is test positive or test negative, wherein the predetermined threshold has a negative predictive value of at least 80%;
c) providing a clinical assessment, wherein if the subject is determined to be test negative, the clinical assessment comprises recommending the subject is excluded from treatment;
d) measuring the amount of at least one third biomarker in at least one third sample and the amount of at least one fourth biomarker in at least one fourth sample from a subject determined to be test positive in step (b) to generate a second score, wherein one of the at least third sample and at least fourth sample is tumor tissue and at least one of the at least third sample and at least fourth sample is normal tissue adjacent to the tumor tissue;
e) comparing the second score to a second predetermined threshold to determine if the subject is test positive for the second predetermined threshold, wherein the predetermined threshold has a positive predictive value of at least 40%; and
f) providing a clinical assessment, wherein if the subject is determined to be test positive in step (e), the clinical assessment comprises recommending the subject receive treatment.


SEQUENCE LISTING (2 sequences)

SEQ ID NO: 1 - DNA, 2298 bp, Homo sapiens
tgtgggtgcggggagtggaattttggaacgaaatgtaacgaagagaagtacagtagtaagagtaacactgtagccgccaccggcaaggggtgcgcgctggggagcggacgctgcatcccctttctgctgcaggaacctctcatcagaccgcctgagggaagcggcgcccggagacccgccccggcccggtccacattctccccaggaagccggactctatggggcgggaccctgggggagcctgagccgagcccggagccagccccgaacccctgaacctccagccaggggcgccccgggagcagccagcccgtgggcgagccgcccgcccgccgagcagccatgagcgagacggtcatctgttccagccgggccactgtgatgctttatgatgatggcaacaagcgatggctccctgctggcacgggtccccaggccttcagccgcgtccagatctaccacaaccccacggccaattcctttcgcgtcgtgggccggaagatgcagcccgaccagcaggtggtcatcaactgtgccatcgtccggggtgtcaagtataaccaggccacccccaacttccatcagtggcgcgacgctcgccaggtctggggcctcaacttcggcagcaaggaggatgcggcccagtttgccgccggcatggccagtgccctagaggcgttggaaggaggtgggccccctccacccccagcacttcccacctggtcggtcccgaacggcccctccccggaggaggtggagcagcagaaaaggcagcagcccggcccgtcggagcacatagagcgccgggtctccaatgcaggaggcccacctgctccccccgctgggggtccacccccaccaccaggacctccccctcctccaggtccccccccacccccaggtttgcccccttcgggggtcccagctgcagcgcacggagcagggggaggaccaccccctgcaccccctctcccggcagcacagggccctggtggtgggggagctggggccccaggcctggccgcagctattgctggagccaaactcaggaaagtcagcaagcaggaggaggcctcaggggggcccacagcccccaaagctgagagtggtcgaagcggaggtgggggactcatggaagagatgaacgccatgctggcccggagaaggaaagccacgcaagttggggagaaaacccccaaggatgaatctgccaatcaggaggagccagaggccagagtcccggcccagagtgaatctgtgcggagaccctgggagaagaacagcacaaccttgccaaggatgaagtcgtcttcttcggtgaccacttccgagacccaaccctgcacgcccagctccagtgattactcggacctacagagggtgaaacaggagcttctggaagaggtgaagaaggaattgcagaaagtgaaagaggaaatcattgaagccttcgtccaggagctgaggaagcggggttctccctgaccacagggacccagaagacccgcttctcctttccgcacacccggcctgtcaccctgctttccctgcctctacttgacttggaattggctgaagactacacaggaatgcatcgttcccactccccatcccacttggaaaactccaagggggtgtggcttccctgctcacacccacactggctgctgattggctggggaggcccccgcccttttctccctttggtccttcccctctgccatccccttggggccggtccctctgctggggatgcaccaatgaaccccacaggaagggggaaggaaggagggaatttcacattcccttgttctagattcactttaacgcttaatgccttcaaagttttggtttttttaagaaaaaaaaatatatatatatttgggttttgggggaaaagggaaatttttttttctctttggttttgataaaatgggatgtgggagtttttaaatgctatagccctgggcttgccccatttggggcagctatttaaggggaggggatgtctcaccgggctgggggtgagatatccccccaccccagggactccccttccctctggctccttccccttttctatgaggaaataagatgctgtaactttttggaacctcagttttttgattttttatttgggtaggttttggggtccaggccattttttttaccccttggaggaaataagatgagggagaaaggagaaggggaggaaacttctcccctcccaccttcacctttagcttcttgaaaatgggcccctgcagaataaatctgccagtttttataaaaaaaaaa

SEQ ID NO: 2 - protein, 380 aa, Homo sapiens
MSETVICSSRATVMLYDDGNKRWLPAGTGPQAFSRVQIYHNPTANSFRVVGRKMQPDQQVVINCAIVRGVKYNQATPNFHQWRDARQVWGLNFGSKEDAAQFAAGMASALEALEGGGPPPPPALPTWSVPNGPSPEEVEQQKRQQPGPSEHIERRVSNAGGPPAPPAGGPPPPPGPPPPPGPPPPPGLPPSGVPAAAHGAGGGPPPAPPLPAAQGPGGGGAGAPGLAAAIAGAKLRKVSKQEEASGGPTAPKAESGRSGGGGLMEEMNAMLARRRKATQVGEKTPKDESANQEEPEARVPAQSESVRRPWEKNSTTLPRMKSSSSVTTSETQPCTPSSSDYSDLQRVKQELLEEVKKELQKVKEEIIEAFVQELRKRGSP